(19) United States
(12) Patent Application Publication
(10) Pub. No.: US 2022/0089655 A1
(43) Pub. Date: Mar. 24, 2022

(54) NOVEL POLYPEPTIDE-MODIFYING ENZYMES AND USES THEREOF

(71) Applicant: ETH ZURICH, Zurich (CH)

(72) Inventors: Jörn PIEL, Zurich (CH); Agneya BHUSHAN, Zurich (CH)

(21) Appl. No.: 17/416,980

(22) PCT Filed: Dec. 16, 2019
(86) PCT No.: PCT/EP2019/085355

§ 371 Date: Jun. 21, 2021

(30) Foreign Application Priority Data
Dec. 19, 2018 (EP) 18213898.2

(51) Int. Cl. C07K 14/22; C12N 9/88; C12N 9/90; C12N 9/10; C12N 15/52

(52) U.S. Cl. CPC C07K 14/22; C12N 9/88; C12Y 201/01107; C12N 9/1007; C12N 15/52; C12N 9/90

(57) ABSTRACT

The present invention is directed to all aspects of novel polypeptide-modifying enzymes from an enzyme cluster in Microvirgula aerodenitrificans. The present invention also relates to nucleic acids encoding these enzymes as well as corresponding vectors and host cells comprising these. Moreover, the present invention encompasses the use of said enzymes in methods for modifying (poly)peptides of interest.

### CROSS-REFERENCE TO RELATED APPLICATIONS

This application is a National Stage of PCT/EP2019/085355, filed 16 Dec. 2019, titled NOVEL POLYPEPTIDE-MODIFYING ENZYMES AND USES THEREOF, published as International Patent Application Publication No. WO 2020/127054, which claims the benefit and priority to European Application No. 18213898.2, filed on 19 Dec. 2018, both of which are incorporated herein by reference in their entirety for all purposes.

### INCORPORATION BY REFERENCE

In compliance with 37 C.F.R. § 1.52(e)(5), the sequence information contained in electronic file name: PCT Sequence Listing st25.txt; size 35.5 KB; created on: 16 Dec. 2019 using Patent-In 3.5 and Checker is hereby incorporated herein by reference in its entirety.

### FIELD OF THE INVENTION

The present invention is directed to all aspects of novel polypeptide-modifying enzymes from an enzyme cluster in Microvirgula aerodenitrificans. The present invention also relates to nucleic acids encoding these enzymes as well as corresponding vectors and host cells comprising these. Moreover, the present invention encompasses the use of said enzymes in methods for modifying (poly)peptides of interest.

### BACKGROUND OF THE INVENTION

Many physiologically active polypeptide-based compounds in nature, for example sponge-related cytotoxins, feature post-translational modifications that have a strong impact on activity. In this respect, marine sponges are a treasure trove of bioactive natural products that exhibit a wide range of activities relevant for biomedical applications. But further development is often impeded by limited supply and synthetically challenging chemical structures. Biological strategies have been proposed for sustainable and economic production based on the suspected or known role of symbiotic bacteria as actual sources of many sponge compounds. However, to date these have not been implemented, mainly because the known producers remain uncultured, are only distantly related to established bacterial hosts for heterologous gene expression, and commonly use unconventional, poorly studied enzymes for natural product biosynthesis.

Among the most complex and biosynthetically unusual natural products known are the polytheonamides from the sponge Theonella swinhoei. These remarkable 49-residue peptides form a β-helical structure and insert into membranes as unimolecular pores, resulting in potent cytotoxicity at lower picomolar range. The chemical basis of this mechanism is the presence of numerous nonproteinogenic residues with lipophilic or other modifications, as well as an almost perfect alternation of d- and l-configured amino acids that is only interrupted by achiral Gly residues.

The polytheonamides with their unusual peptide structure are of ribosomal biosynthetic origin and belong to a new family of ribosomally synthesized and post-translationally modified peptides (RiPPs), termed proteusins.

It was found that when acting on PoyA, a precursor protein comprised of standard l-amino acids, only seven enzymes introduce a total of 50 posttranslational modifications in a highly promiscuous but precisely controlled fashion (Freeman et al. Nat. Chem. 9, 387-395, 2017). Like most other RiPPs (Arnison et al., Nat Prod Rep 30, 108-60, 2016), PoyA is organized into an N-terminal leader region and a C-terminal core that is post-translationally modified and ultimately released from the leader. As the earliest-acting modifying enzyme, one radical S-adenosylmethionine (rSAM) enzyme, PoyD, generates 18 D-amino acids by epimerization of the PoyA core. Further iterative enzymes install 8 N-methylations of Asn side chains (PoyE), 4 hydroxylations (PoyI), 1 dehydration at Thr (PoyF), and 17 methylations at diverse non-activated carbon atoms (PoyB and PoyC), including 4 methylations that together create a t-butyl unit (PoyC). Ultimately, proteolytic cleavage by PoyH releases the core and triggers hydrolysis of an N-terminal enamine function at the t-butylated Thr to generate the pharmacologically important α-keto moiety of polytheonamides.

Considerable challenges were encountered when attempting to reconstitute the complete enzymatic pathway in heterologous bacterial hosts.

For example, although the epimerase PoyD acts irreversibly at each amino acid center, its co-production with PoyA in the bacterial host E. coli resulted in mixtures of peptide products that were only processed at the C-terminal half (Freeman et al., Nat. Chem. 9, 387-395, 2017). The most recalcitrant enzymes were the C-methyltransferases PoyB and PoyC, which remained completely inactive in E. coli. Both are cobalamin-dependent rSAM methyltransferases, a highly challenging protein family in the context of biotechnological applications (Lanz et al., Biochemistry 57, 1475-1490, 2018). Functional expressions of poyB and poyC were ultimately successful in the non-standard host Rhizobium leguminosarum (Freeman et al., Nat. Chem. 9, 387-395, 2017), which unlike E. coli contains a complete cobalamin biosynthetic pathway (Burton et al., Canadian Journal of Botany 30, 521-524, 1952). In this way, C-methylations occurred at most of the core positions, but with low efficiency and resulting in complex mixtures of mono- to tetra-methylated products.

Due to the challenges of identifying and expressing genes from invertebrate symbionts, biological synthesis has to date only been achieved for a single example, patellamide-type RiPPs from tunicate-associated cyanobacteria (Donia et al., Nat Chem Biol 2, 729-35, 2006).

Cobalamin (vitamin B12)-dependent radical S-adenosyl methionine (Cbl-dependent rSAM) enzymes catalyze some of the synthetically most challenging reactions, such as methylations of unactivated carbon centers. These proteins comprise a large superfamily of currently about 7000 known members (Bridwell-Rabb et al. Nature 544, 322-326 2017). Among the numerous examples of bacteria-derived bioactive natural products generated by such reactions (Huo et al. Chem Biol 19, 1278-1287, 2012) are the economically important carbapenems, thiostrepton, gentamicin, fosfomycin-type compounds, moenomycin, which are all commercial antibiotics. Heterologous efforts to produce such compounds either for overproduction or biosynthetic studies have, however, been limited to organisms of related strains. Some examples of these are fosfomycin (Woodyer et al. Chem Biol 13(11): 1171-1182, 2006), bottromycins and thiostreptons (Huo et al. Chem Biol 19, 1278-1287, 2012, Li et al. Mol. BioSyst. 2011, 7, 82-90), all produced by Streptomyces species, where responsible clusters were transferred into a standard Streptomyces strain to produce these compounds. The Cbl-dependent rSAM methyltransferases involved in these cases, however, catalyze only up to two methylations.

### BRIEF SUMMARY OF THE INVENTION

It is the objective of the present invention to provide new enzymatic tools for the post-translational modification of polypeptides of interest, optionally for use in heterologous hosts, in particular in bacterial hosts, e.g. such as E. coli. Preferably, these enzyme tools catalyze at least one, optionally multiple C-methylations, N-methylations, epimerizations and/or dehydration(s) and optionally lead to homogenous product mixtures. These enzyme tools may have utility for preparing post-translationally modified physiologically active polypeptides, e.g. polypeptide antibiotics, polypeptide cytotoxins, polytheonamides, etc.

In a first aspect, the objective technical problem is solved by an isolated and purified nucleic acid, comprising or consisting of a nucleic acid sequence selected from the group consisting of:

(i) a nucleic acid sequence listed in any one of SEQ ID NOs: 1 (aerC), 3 (aerD), and 5 (aerF), 7 (aerE);
(ii) a nucleic acid sequence of at least 80% or 90% sequence identity, optionally at least 95% or 98% sequence identity with a nucleic acid sequence of (i), optionally over the whole sequence;
(iii) a nucleic acid sequence that hybridizes to the nucleic acid sequence of (i) or (ii) under stringent conditions;
(iv) a fragment of any of the nucleic acid sequences of (i) to (iii), that hybridizes to the nucleic acid sequence of (i) or (ii) under stringent conditions;
(v) a nucleic acid sequence degenerated with respect to the nucleic acid sequence of any of (i) to (iv);
(vi) a nucleic acid sequence, wherein said nucleic acid sequence is derivable by substitution, addition and/or deletion of at least one nucleic acid of the nucleic acid sequences of (i) to (v) that hybridizes to a nucleic acid sequence of (i) or (ii) under stringent conditions;
(vii) a nucleic acid sequence complementary to the nucleic acid sequence of any of (i) to (vi); wherein the nucleic acid sequence of any of (i) to (vii),
(a) when based on SEQ ID NO: 1 (aerC) encodes a polypeptide that has cobalamin-dependent rSAM methyltransferase activity, optionally methylates one or more valine(s) to tert-leucine(s), methylates one or more isoleucine(s), methylates one or more leucine(s) and/or methylates one or more threonine(s);
(b) when based on SEQ ID NO: 3 (aerD) encodes a polypeptide that has rSAM epimerase activity to convert one or more L-amino acid(s) into D-amino acid(s);
(c) when based on SEQ ID NO: 5 (aerF) encodes a polypeptide that has dehydratase activity to dehydrate an N-terminal threonine and serine to an alpha-keto functional group; or
(d) when based on SEQ ID NO: 7 (aerE) encodes a polypeptide that has asparagine (ASN)N-methyltransferase activity for methylating one or more side chain amines of one or more asparagine(s).

It was surprisingly found that the above nucleic acids derived from Microvirgula denitrificans encode fully functional enzymes that modify polypeptides of interest in a stable, efficient, and often in a repetitive manner, i.e. multiple modifications of the same polypeptide substrate, and which enzymes produce homogenous products. And even more surprisingly, the enzymes can function in heterologous organisms such as bacteria, e.g. E. coli.

The term “% (percent) sequence identity” as known to the skilled artisan and used herein in the context of nucleic acids indicates the degree of relatedness among two or more nucleic acid molecules that is determined by agreement among the sequences. The percentage of “sequence identity” is the result of the percentage of identical regions in two or more sequences while taking into consideration the gaps and other sequence peculiarities.

The identity of related nucleic acid molecules can be determined with the assistance of known methods. In general, special computer programs are employed that use algorithms adapted to accommodate the specific needs of this task. Preferred methods for determining identity begin with the generation of the largest degree of identity among the sequences to be compared. Preferred computer programs for determining the identity among two nucleic acid sequences comprise, but are not limited to, BLASTN (Altschul et al., (1990) J. Mol. Biol., 215:403-410) and LALIGN (Huang and Miller, (1991) Adv. Appl. Math., 12:337-357). The BLAST programs can be obtained from the National Center for Biotechnology Information (NCBI) and from other sources (BLAST handbook, Altschul et al., NCB NLM NIH Bethesda, Md. 20894).

The nucleic acid molecules according to the invention may be prepared synthetically by methods well-known to the skilled person, but also may be isolated from suitable DNA libraries and other publicly available sources of nucleic acids and subsequently may optionally be mutated. The preparation of such libraries or mutations is well-known to the person skilled in the art.

The nucleic acid of the present invention may be a DNA, RNA or PNA, optionally DNA or PNA.

In some instances, the present invention also provides novel nucleic acids encoding the polypeptide enzymes of the present invention characterized in that they have the ability to hybridize to a specifically referenced nucleic acid sequence, optionally under stringent conditions. Next to common and/or standard protocols in the prior art for determining the ability to hybridize to a specifically referenced nucleic acid sequence under stringent conditions (e.g. Sambrook and Russell, (2001) Molecular cloning: A laboratory manual (3 volumes)), it is preferred to analyze and determine the ability to hybridize to a specifically referenced nucleic acid sequence under stringent conditions by comparing the nucleotide sequences, which may be found in gene databases (e.g. www.ncbi.nlm.nih.gov/entrez/query.fcgi?db=nucleotide and genome.jgi.doe.gov/programs/fungi/index.jsf) with alignment tools, such as e.g. the abovementioned BLASTN (Altschul et al., (1990) J. Mol. Biol., 215:403-410), LALIGN alignment tools and multiple alignment tools such as e.g. CLUSTALW (Sievers F et al., (2011) Mol. Sys. Bio. 7: 539), MUSCLE (Edgar., (2004) Nucl. Acids Res. 32:1792-7) or T-COFFEE (Notredame et al., (2000) J of Mol. Bio 302 1: 205-17).

Most preferably, the ability of a nucleic acid of the present invention to hybridize to a specifically referenced nucleic acid, e.g. those listed in any of SEQ ID NOs 1, 3, 5 and 7, is confirmed in a Southern blot assay under the following conditions: 6× sodium chloride/sodium citrate (SSC) at 45° C. followed by a wash in 0.2×SSC, 0.1% SDS at 65° C.

The term “nucleic acid encoding a polypeptide” as used in the context of the present invention is meant to include allelic variations and redundancies in the genetic code. For example, the term “a nucleic acid sequence degenerated with respect to the nucleic acid code” in the context of a specific nucleic acid sequence, e.g. SEQ ID NOs: 1, 3, 5 or 7, is meant to describe nucleic acids that differ from the specified sequence but encode the identical amino acid sequence.

The nucleic acids of the present invention code for specific polypeptide enzymes, in particular,
(a) a polypeptide that has cobalamin-dependent rSAM methyltransferase activity, optionally methylates one or more valine(s) to tert-leucine(s), methylates one or more isoleucine(s), methylates one or more leucine(s) and/or methylates one or more threonine(s);
(b) a polypeptide that has rSAM epimerase activity to convert one or more L-amino acid(s) into D-amino acid(s);
(c) a polypeptide that has dehydratase activity to dehydrate an N-terminal threonine or serine to an alpha-keto functional group; or
(d) a polypeptide that has asparagine (ASN)N-methyltransferase activity for methylating one or more side chain amines of one or more asparagine(s).

Therefore, in a further aspect, the invention relates to an isolated and purified polypeptide selected from the group consisting of:
(i) polypeptides comprising or consisting of an amino acid sequence selected from the group consisting of SEQ ID NOs: 2, 4, 6 and 8,
(ii) polypeptides encoded by any of the nucleic acids of claim 1;
(iii) polypeptides having an amino acid sequence identity of at least 70% or 80%; optionally at least 90% or 95% with the polypeptides of (i) and/or (ii); and
(iv) a functional fragment and/or functional derivative of (i), (ii) or (iii);
   wherein the polypeptide of any of (i) to (iv),
   (a) when based on an amino acid sequence of SEQ ID NO: 2 (AerC) has cobalamin-dependent rSAM methyltransferase activity, optionally methylates one or more valine(s) to tert-leucine(s), methylates one or more isoleucine(s), methylates one or more leucine(s) and/or methylates one or more threonine(s);
   (b) when based on an amino acid sequence of SEQ ID NO: 4 (AerD) has rSAM epimerase activity to convert one or more L-amino acid(s) into D-amino acid(s);
   (c) when based on an amino acid sequence of SEQ ID NO: 6 (AerF) has dehydratase activity to dehydrate an N-terminal threonine or serine to an alpha-keto functional group; or
   (d) when based on an amino acid sequence of SEQ ID NO: 8 (AerE) has asparagine (ASN)N-methyltransferase activity for methylating one or more side chain amine(s) of asparagine(s).

The term “when based on” in conjunction with a specified amino acid sequence indicates that the polypeptide is one of the polypeptides defined in any of passages (i) to (iv) above.

The term (poly)peptide, as used herein, is meant to encompass peptides, polypeptides, oligopeptides and proteins that comprise two or more amino acids linked covalently through peptide bonds. The term does not refer to a specific length of the product. Optionally, the term (poly)peptide includes (poly)peptides with post-translational modifications, for example, glycosylations, acetylations, phosphorylations and the like, as well as (poly)peptides comprising non-natural or non-conventional amino acids and functional derivatives as described below. The term non-natural or non-conventional amino acid refers to naturally occurring or naturally not occurring unnatural amino acids or chemical amino acid analogues, e.g. D-amino acids, α,α-disubstituted amino acids, N-alkyl amino acids, homo-amino acids, dehydroamino acids, aromatic amino acids (other than phenylalanine, tyrosine and tryptophan), and ortho-, meta- or para-aminobenzoic acid. Non-conventional amino acids also include compounds which have an amine and carboxyl functional group separated in a 1,3 or larger substitution pattern, such as β-alanine, γ-amino butyric acid, Freidinger lactam, the bicyclic dipeptide (BTD), amino-methyl benzoic acid and others well known in the art. Statine-like isosteres, hydroxyethylene isosteres, reduced amide bond isosteres, thioamide isosteres, urea isosteres, carbamate isosteres, thioether isosteres, vinyl isosteres and other amide bond isosteres known to the art may also be used.

The percentage identity of related amino acid molecules can be determined with the assistance of known methods. In general, special computer programs are employed that use algorithms adapted to accommodate the specific needs of this task. Preferred methods for determining identity begin with the generation of the largest degree of identity among the sequences to be compared. Preferred computer programs for determining the identity among two amino acid sequences comprise, but are not limited to, TBLASTN, BLASTP, BLASTX, TBLASTX (Altschul et al., J. Mol. Biol., 215, 403-410, 1990), or ClustalW (Larkin M A et al., Bioinformatics, 23, 2947-2948, 2007). The BLAST programs can be obtained from the National Center for Biotechnology Information (NCBI) and from other sources (BLAST handbook, Altschul et al., NCB NLM NIH Bethesda, Md. 20894). The ClustalW program can be obtained from www.clustal.org.

The term “functional derivative” of a (poly)peptide of the present invention is meant to include any (poly)peptide or fragment thereof that has been chemically or genetically modified in its amino acid sequence, e.g. by addition, substitution and/or deletion of amino acid residue(s) and/or has been chemically modified in at least one of its atoms and/or functional chemical groups, e.g. by additions, deletions, rearrangement, oxidation, reduction, etc. as long as the derivative still has at least some enzymatic activity to a measurable extent, e.g. of at least about 1 to 10%, preferably 10 to 50% enzymatic activity of the original unmodified (poly)peptide of the invention.

In this context a “functional fragment” of the invention is one that forms part of a (poly)peptide or derivative of the invention and still has at least some enzymatic activity to a measurable extent, e.g. of at least about 1 to 10%, preferably 10 to 50% enzymatic activity of the original unmodified (poly)peptide of the invention.

The enzymatic polypeptides of the present invention can be used to modify substrate polypeptides with broad amino acid sequence variation. The enzymes can be isolated or partially purified before use. Specific antibodies can be used to identify, isolate, purify, localise or bind the enzymes of the present invention.

Therefore, in a further aspect the present invention also reads on an antibody, optionally a monoclonal antibody, a functional fragment or functional derivative thereof, or antibody-like binding protein that specifically binds a polypeptide of the invention.

Antibodies, functional fragments and functional derivatives thereof for practicing the invention are routinely available by hybridoma technology (Kohler and Milstein, Nature 256, 495-497, 1975), antibody phage display (Winter et al., Annu. Rev. Immunol. 12, 433-455, 1994), ribosome display (Schaffitzel et al., J. Immunol. Methods, 231, 119-135, 1999) and iterative colony filter screening (Giovannoni et al., Nucleic Acids Res. 29, E27, 2001) once the target antigen is available. Typical proteases for fragmenting antibodies into functional products are well-known. Other fragmentation techniques can be used as well as long as the resulting fragment has a specific high affinity and, preferably a dissociation constant in the micromolar to picomolar range.

A very convenient antibody fragment for targeting applications is the single-chain Fv fragment, in which a variable heavy and a variable light domain are joined together by a polypeptide linker. Other antibody fragments for vascular targeting applications include Fab fragments, Fab2 fragments, miniantibodies (also called small immune proteins), tandem scFv-scFv fusions as well as scFv fusions with suitable domains (e.g. with the Fc portion of an immuneglobulin). For a review on certain antibody formats, see Holliger P, Hudson Pt; Engineered antibody fragments and the rise of single domains. Nat Biotechnol. 2005 September, 23(9):1126-36.).

The term “functional derivative” of an antibody for use in the present invention is meant to include any antibody or fragment thereof that has been chemically or genetically modified in its amino acid sequence, e.g. by addition, substitution and/or deletion of amino acid residue(s) and/or has been chemically modified in at least one of its atoms and/or functional chemical groups, e.g. by additions, deletions, rearrangement, oxidation, reduction, etc. as long as the derivative has substantially the same binding affinity as to its original antigen and, preferably, has a dissociation constant in the micro-, nano- or picomolar range. A most preferred derivative of the antibodies for use in the present invention is an antibody fusion protein that will be defined in more detail below.

In a preferred embodiment, the antibody, fragment or functional derivative thereof for use in the invention is one that is selected from the group consisting of polyclonal antibodies, monoclonal antibodies, chimeric antibodies, humanized antibodies, CDR-grafted antibodies, Fv-fragments, Fab-fragments and Fab2-fragments and antibody-like binding proteins, e.g. affilines, anticalines and aptamers.

For a review of antibody-like binding proteins see Binz et al. on engineering binding proteins from non-immunoglobulin domains in Nature Biotechnology, Vol. 23, No. 10, October 2005, 12571268. The term “aptamer” describes nucleic acids that bind to a polypeptide with high affinity. Aptamers can be isolated from a large pool of different single-stranded RNA molecules by selection methods such as SELEX (see, e.g., Jayasena, Clin. Chem., 45, p. 1628-1650, (1999); Klug and Famulok, M. Mol. Biol. Rep., 20, p. 97-107 (1994); U.S. Pat. No. 5,582,981). Aptamers can also be synthesized and selected in their mirror form, for example, as the L-ribonucleotide (Nolte et al., Nat. Biotechnol., 14, pp. 1116-1119, (1996); Klussmann et al., Nat. Biotechnol., 14, p. 1112-1115, (1996)). Forms isolated in this way have the advantage that they are not degraded by naturally occurring ribonucleases and, therefore, have a greater stability.

Another antibody-like binding protein and alternative to classical antibodies are the so-called “protein scaffolds”, for example, anticalines, that are based on lipocaline (Beste et al., Proc. Natl. Acad. Sci. USA, 96, p. 1898-1903, (1999)). The natural ligand binding sites of lipocalines, for example, of the retinol-binding protein or bilin-binding protein, can be changed, for example, by employing a “combinatorial protein design” approach, and in such a way that they bind selected haptens (Skerra, Biochem. Biophys. Acta, 1482, pp. 337-350, (2000)). For other protein scaffolds it is also known that they are alternatives for antibodies (Skerra, J. Mol. Recognit, 13, pp. 167-287, (2000)). (Hey, Trends in Biotechnology, 23, pp. 514-522, (2005)).

According to the invention the term functional antibody derivative is meant to include said protein-derived alternatives for antibodies, i.e. antibody-like binding proteins, e.g. affilines, anticalines and aptamers that specifically recognize at least one extracellular domain of oncofetal fibronectin or oncofetal tenascin.

In summary, the terms antibody, functional fragment and functional derivative thereof denote all substances that have the same or similar specific binding affinity to any one of the extracellular domains of oncofetal fibronectin or oncofetal tenascin as a complete antibody having specific binding affinity to these targets.

The polypeptide enzymes of the present invention may be encoded and expressed by a vector, optionally a bacterial plasmid, comprising a nucleic acid of the present invention and optionally nucleic acids further encoding and expressing a polypeptide of interest for posttranslational modification by at least one enzymatic polypeptide of the present invention.

For example, vectors suitable for practicing the present invention may be selected from the group of vectors consisting of pLMB509, pLMB51, pK18mobSacB, pET 28b, pACYC DUET, pCDF DUET, pET DUET, pRSF DUET and pBAD vectors.

Unlike other sponge-related post-translationally modifying enzymes the enzymes of the present invention can be transferred functionally into bacterial host cells and stably and efficiently produce homogeneously modified polypeptides of interest.

In this regard, the present invention also provides for a bacterial host cell, optionally a bacterial host cell producing cobalamin, optionally Microvirgula aerodenitrificans or E. coli host cell, optionally a cobalamin-producing E. coli, comprising at least one or more of the nucleic acids of the present invention, wherein the host cell expresses and modifies a heterologous polypeptide of interest by one or more polypeptides of the present invention. For example, the host cell for practicing the present invention may be selected from the group consisting of Microvirgula sp. AG722, Microvirgula aerodenitrificans strain BE2.4, Microvirgula curvata, Microvirgula sp. DB2-7, Microvirgula sp. H8, Microvirgula sp. HW7, Cystobacter fucus, Rhizobium leguminosarum and Sinorhizobium meliloti.

Even though the enzymes of the present invention exist naturally in Microvirgula aerodenitrificans, it was so far only speculated that this organism may actually express enzymes, for example, with cobalamin-dependent rSAM methyltransferase activity. And this assumption was based on the finding of sequence analogies only. Based on sequence analogy, these rSAM proteins comprise a large superfamily of currently about 7000 known members (Bridwell-Rabb et al. Nature 544, 322-326 2017). However, their activity, transferability into heterologous organisms, their substrate specificity and their ability to produce homogenous products differs widely.

In a further embodiment, the present invention relates to a Microvirgula aerodenitrificans host cell, wherein the host cell expresses at least one heterologous polypeptide for enzymatic modification and one or more polypeptides of the present invention, thereby modifying the at least one heterologous polypeptide by the one or more polypeptides. The Microvirgula aerodenitrificans host cell of the present invention clearly differs from the naturally occurring Microvirgula aerodenitrificans by the heterologous substrate polypeptide. This difference can be easily verified by sequence comparison of the nucleic acid sequences of a naturally occurring Microvirgula aerodenitrificans with the corresponding sequences of a recombinantly modified Microvirgula aerodenitrificans host cell. Alternatively, and when antibodies for the heterologous protein are available, both host cells can by lysed and the antibodies can be applied to specifically bind and identify the heterologous polypeptide.

For example, a Microvirgula aerodenitrificans host cell of the present invention may express

(i) at least one polypeptide based on amino acid sequence SEQ ID NO: 2 (AerC);
(ii) at least one polypeptide based on amino acid sequence SEQ ID NO: 4 (AerD);
(iii) at least one polypeptide based on amino acid sequence SEQ ID NO: 6 (AerF); and/or
(iv) at least one polypeptide based on amino acid sequence SEQ ID NO: 8 (AerE),
(v) with the proviso that expression of polypeptide (iv) requires the expression of polypeptide (ii).

It was found that the enzymatic activity of an asparagine N-methyltransferase based on SEQ ID NO: 8 requires the co-existence of an rSAM epimerase based on SEQ ID NO: 4. In another embodiment the host cell of the invention expresses at least the polypeptide of (iv) based on SEQ ID NO: 8 and at least the polypeptide of (ii) based on SEQ ID NO: 4).

In a further embodiment, the bacterial host cell of the present invention is an Escherichia coli host cell expressing

(i) at least one polypeptide based on amino acid sequence SEQ ID NO: 2 (AerC);
(ii) at least one polypeptide based on amino acid sequence SEQ ID NO: 4 (AerD);
(iii) at least one polypeptide based on amino acid sequence SEQ ID NO: 6 (AerF)2; and/or
(iv) at least one polypeptide based on amino acid sequence SEQ ID NO: 8 (AerE), with the proviso that (a) expression of polypeptide (iv) requires the expression of polypeptide (ii) and (b) expression of (i) requires bacterial production or supplement of cobalamin (preferably D and E).

The host cells of the present invention are particularly useful for preparing polypeptide-based antibiotics from polypeptide precursor substrates that can be produced recombinantly. For example, host cells of the present invention may be a Microvirgula aerodenitrificans or Escherichia coli host cell expressing a heterologous polypeptide for enzymatic modification selected from the group of polypeptide precursors of boceprevir, telapevir, glecaprevir, atazanavir, vancomycin, colistin, teixobactin, bacitracin, gramicidin A-D, goserelin, leuprolide, nateglidine, octreotide, thiostreptons, bottromycins, polymyxin, actinomycin, nisin, protegrin, dalbavancin, daptomycin, enfurvirtide, oritavancin, teicoplanin, and guavanin 2.

In wild type Microvirgula aerodenitrificans the natural substrate forms part of the AerA cluster together with a leader sequence featuring nucleic acid sequence SEQ ID NO: 9 and corresponding amino acid sequence SEQ ID NO: 10, which directs the substrate to the cytosol.

The invention also encompasses a Microvirgula aerodenitrificans, optionally an Escherichia coli host cell of the invention expressing a heterologous polypeptide for enzymatic modification encoded by a nucleic acid sequence comprised in the aerA cluster and encompassing the nucleic acid sequence of Seq. ID. NO.: 9 or a nucleic acid sequence hybridizing thereto under stringent conditions.

The present invention is also directed to a composition comprising at least one nucleic acid, at least one polypeptide, at least one vector, or at least one bacterial host cell of the present invention as described herein.

The nucleic acid sequences, amino acid sequences, vectors and host cells of the present invention have utility for use in a method for producing and modifying a heterologous polypeptide in a Microvirgula aerodenitrificans cell or an E. coli cell, optionally a cobalamin-producing E. coli cell. For example, such method may comprise the steps of
(i) providing a Microvirgula aerodenitrificans or E. coli host cell of the invention, optionally a cobalamin-producing E. coli host cell, functionally expressing
   a. at least one polypeptide enzyme of the invention and
   b. at least one heterologous polypeptide of interest; and
(ii) co-expressing the at least one polypeptide enzyme of the invention and the at least one heterologous polypeptide of interest;
(iii) and optionally co-expressing one or more further enzymes for modifying the at least one heterologous polypeptide of interest;
(iv) and optionally at least partially purifying the so-modified heterologous polypeptide,
(v) wherein the at least one polypeptide enzyme of the invention is capable of catalyzing at least one modification in the heterologous polypeptide.

Optionally, the method of the invention may comprise the steps of
(i) providing a Microvirgula aerodenitrificans or a cobalamin-producing E. coli host cell, functionally expressing
   a. at least one Cbl-dependent rSAM polypeptide enzyme of the invention and
   b. at least one heterologous polypeptide of interest; and
(ii) co-expressing the at least one Cbl-dependent rSAM enzyme and the at least one heterologous polypeptide;
(iii) and optionally co-expressing one or more further enzymes for modifying the heterologous polypeptide of interest;
(iv) and optionally at least partially purifying the so-modified heterologous (poly)peptide of interest; wherein the at least one Cbl-dependent rSAM enzyme methylates one or more valine(s) to tert-leucine(s), methylates one or more isoleucine(s), methylates one or more leucine(s) and/or methylates one or more threonine(s) in the at least one heterologous polypeptide of interest.

For the above methods it is optional that the one or more further enzymes for modifying the heterologous polypeptide(s) in step (iii) are selected from the polypeptides of the invention.

In a further embodiment of the invention the method is one, wherein the one or more further enzymes for modifying the heterologous polypeptide(s) in step (iii) are selected from the group consisting of PoyB, PoyC (rSAM C-methyltransferases, Freeman et al., Nat. Chem. 9, 387-395, 2017), OspD, AvpD, PlpD, PoyD (Epimerases, Morinaka et. al. Angewandte Chemie, 56(3): 762-766, 2017), PlpXY (β-amino acid incorporation, Morinaka et. al. Science, 359, 779, 2018,), PtsY (S-methyltransferase, Helf et. al., Chem Bio Chem 18:444-450, 2017).

The method of the present invention is specifically suited for preparing polypeptide antibiotics from polypeptide precursors thereof.

For example, the methods of the invention can be used for modifying at least one heterologous polypeptide selected from the group consisting of polypeptide precursors of boceprevir, telapevir, glecaprevir, atazanavir, vancomycin, colistin, teixobactin, bacitracin, gramicidin A-D, goserelin, leuprolide, nateglidine, octreotide, thiostreptons, bottromycins polymyxin, actinomycin, nisin, protegrin, dalbavancin, daptomycin, enfurvirtide, oritavancin, teicoplanin and guavanin 2.

Another example for practicing the present invention is a method, wherein at least one, two or all of (i) the heterologous polypeptide(s) of interest, the polypeptide enzyme(s) of the present invention and/or the one or more further enzymes for modifying the heterologous polypeptide(s) are present in the form of host-integrated DNA and/or in the form of a plasmid.

The invention also relates to the products that are available for the first time with the enzymes, vectors, host cells and methods of the present invention.

For example, the invention encompasses a polypeptide, optionally a cytotoxin, an antibiotic polypeptide or antiviral polypeptide comprising a posttranslational modification selected from the group consisting of
(i) a methylation of one or more valine(s) to tert-leucine(s), a methylation of one or more isoleucine(s), a methylation of one or more leucine(s), a methylation of one or more threonine(s);
(ii) a conversion of one or more L-amino acid(s) into D-amino acid(s);
(iii) a hydrolyzation of an N-terminal dehydro-threonine or -serine to an alpha-keto functional group; and
(iv) a methylation of one or more side chain amine(s) of asparagine(s),
   wherein the polypeptide is obtained by a method of any of claims 12 to 17.

In this regard, the invention also pertains to the use of a nucleic acid, a polypeptide, an antibody, a vector, a host cell, composition or a method of the invention as described herein for modifying a heterologous polypeptide in a bacterial host cell or bacterially derived cell-free system.

The following Figures and Examples serve to illustrate the invention and are not intended to limit the scope of the invention as described in the appended claims.

### BRIEF DESCRIPTION OF THE DRAWINGS

FIGS. 1a, 1b, and 1c show the structure and biosynthetic gene cluster of polytheonamides. (1a) Secondary structures of polytheonamide A and B are shown, differing in the configuration of the methionine sulfoxide. Modifications occurring post-translationally on the canonical proteingenic amino acids are shown in the legend below. (1b) Graphical representation of the polytheonamide (poy) gene cluster and comparison with similar candidate clusters (aer, rhp and vep) in other bacteria. Highlighting corresponds to the modifications hypothesized to occur from the encoded ORFs. (1c) Alignment of the core sequences from all clusters. Asn residues predicted to be methylated are underlined, with predicted helix clamps shown.

PoyaA a.a., Candidatus Entotheonella factor
(SEQ ID NO: 11)
QAAGGTGIGVVVAVVAGAVANTGAGVNQVAGGNINVVGNINVNANVSVNM
NQTT
AerA a.a., Microvirgula aerodenitrificans
(SEQ ID NO: 12)
AVAPQTIAVVLVAVVGAAAAAVVTYLGAANVVGAANGTVTANAVANTNAV
A
RhoA1 a.a., Rhodospirillaceae bacterium BRH_c57
(SEQ ID NO: 13)
AVAPQTIAVVVAVVGIGVVAGNTLGVVNNVGAGNAVAAGNVATTGNAVAN
TNVIA
RhoA2 a.a., Rhodospirillaceae bacterium BRH_c57
(SEQ ID NO: 14)
AVAPQTIAVVVAALGVVVANTLGAVNNVGAGNAVTVGNVATTGNAVANST
SVS
RhoA3 a.a., Rhodospirillaceae bacterium BRH_c57
(SEQ ID NO: 15)
AVAPQTIAVVTNGVGVCAVVTGPVTIAYPTNVVTCVVA
VerA a.a., Verrucomicrobia bacterium SCGC
AAA164-I21
(SEQ ID NO: 16)
AVAGGVAAIAVFVVGVVAVAVGGTVTVAVNINAAVNVHTVVNAVKGANES
PW

FIGS. 2a, 2b and 2c show the (2a) extracted ion chromatogram (EIC) looking for the protected AerA core following proteinase K digestion of Nhis-AerA and Nhis AerAD purified from E. coli; (2b) ion of protected core fragment (top) after expressions in H₂O compared with ion observed following the same treatment after ODIS expressions (below). A mass shift of 21 Da was observed that was localized to the residues indicated (2c). Assumed modifications refer to modifications that could not be localized to a particular residue during MS² analysis, but for which fragmentation supported the existence of such a modification.

Nhis-AerA a.a. Microvirgula aerodenitrificans
(SEQ ID NO: 17)
TIAVVLVAVVGAAAAAVVTYLGAANVVGAANGTVTANAVANTNAVA

FIGS. 3a, 3b, and 3c show the conditions for aer expression: (3a) Results of the GusA assay in M. aerodenitrificans. Deeper blue color corresponds to stronger activity of the promoter, with TB-30 (dotted box) the best condition observed. LB—luria bertani medium, TB—terrific broth, NB—nutrient broth. 30 and 37 correspond to temperature at which growth occurred. (3b) EIC following cell-free assays showing product peak for GluC treated Nhis-AerA (5 methylations) and the corresponding mass spectra. (3c) Position of methylations to Asn residues of the core based on MS-MS data. Methylation on Asn43 was not localized but proposed based on y-ion fragment masses observed. (SEQ ID NO: 12).

FIGS. 4a and 4b show the aeronamide characterization from expression in M. aerodenitrificans.

(4a) Total ion chromatogram (TIC) of GluC treated Nhis-AerA(GG). A: major product, B: minor product.

AerA(GG) a.a. Microvirgula aerodenitrificans
(SEQ ID NO: 18)
AVAGGTIAVVLVAVVGAAAAAVVTYLGAANVVGAANGTVTANAVANTNA
VA

(4b) TIC of GluC treated Nhis-AerA. A: major product, B: minor product. Modifications localized to residues as described in the legend.

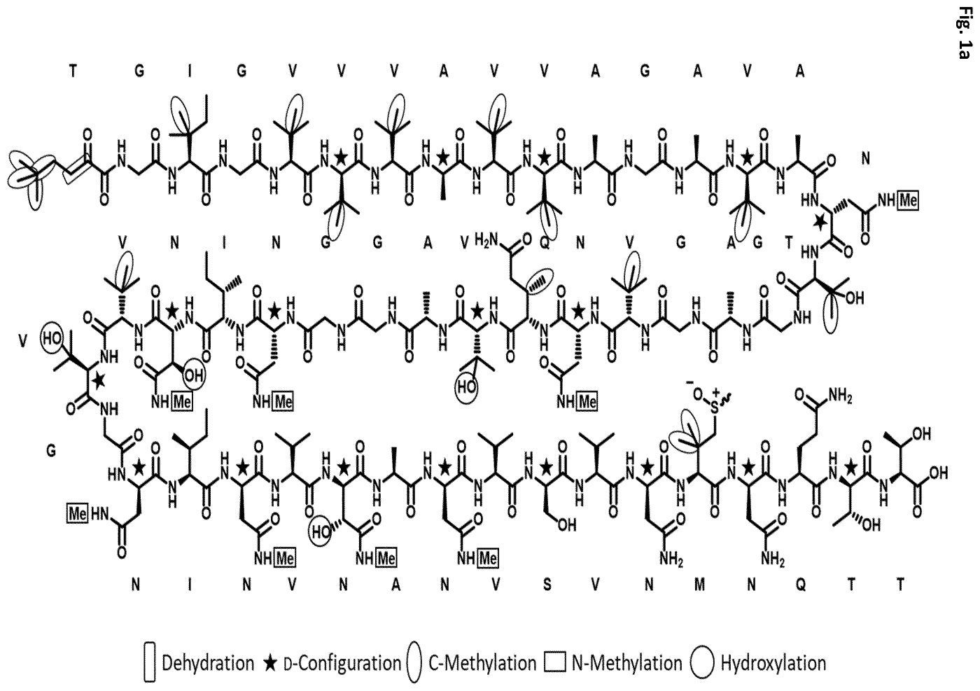

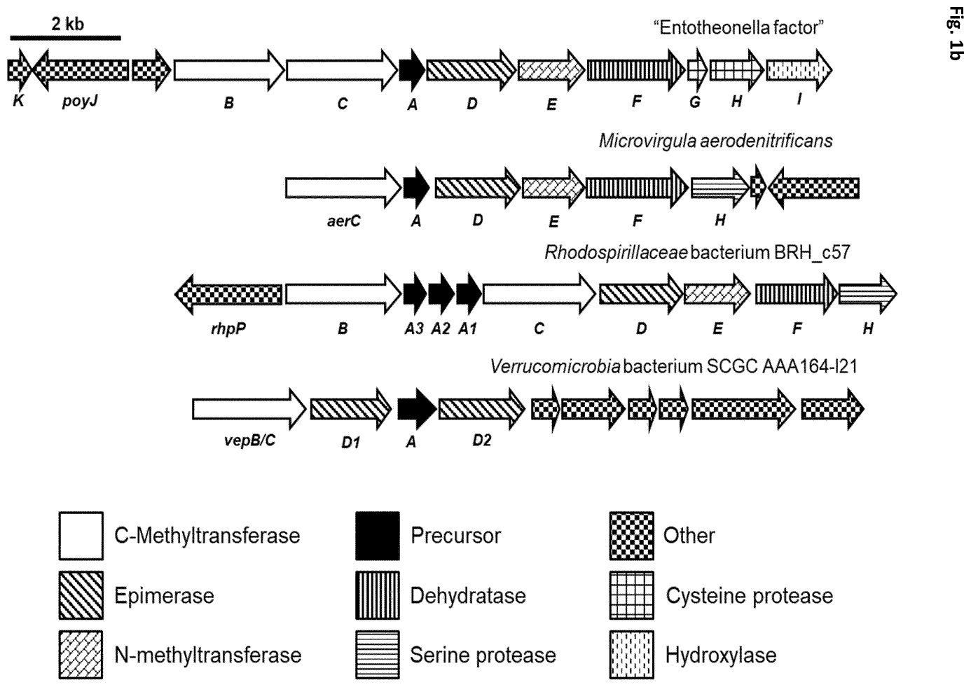

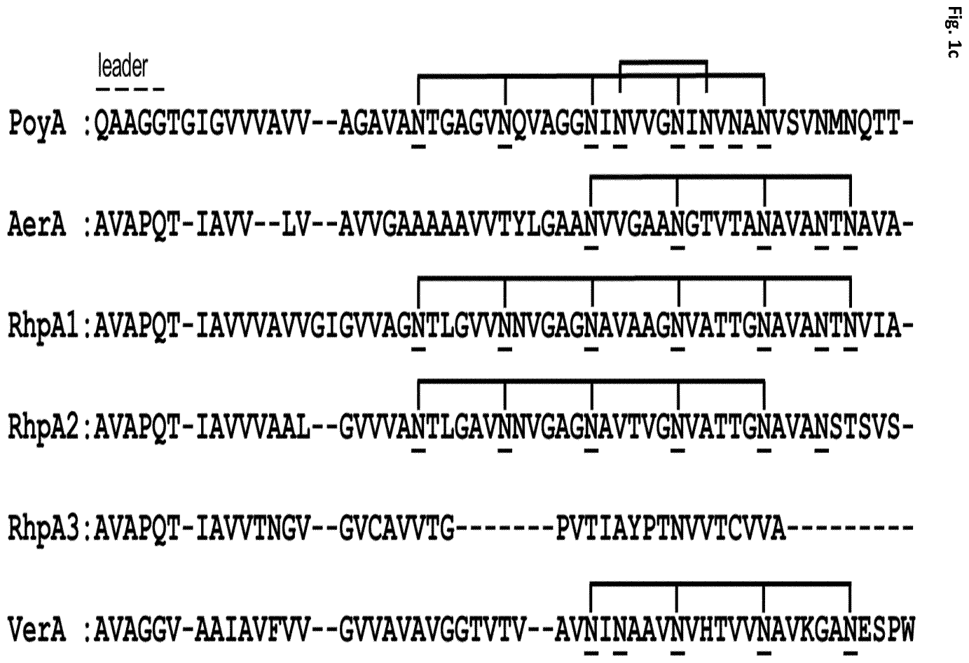

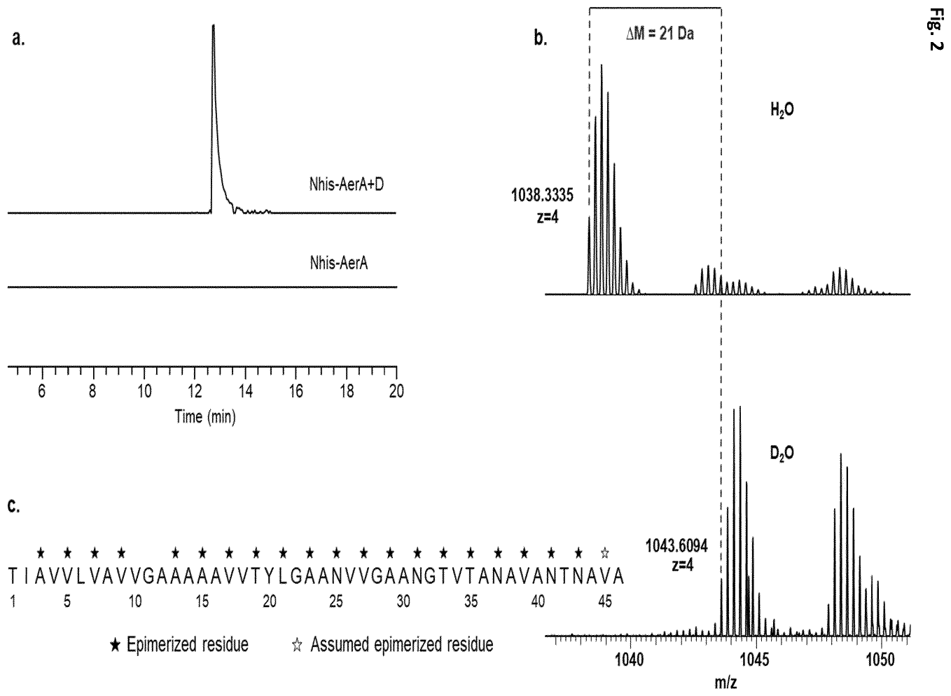

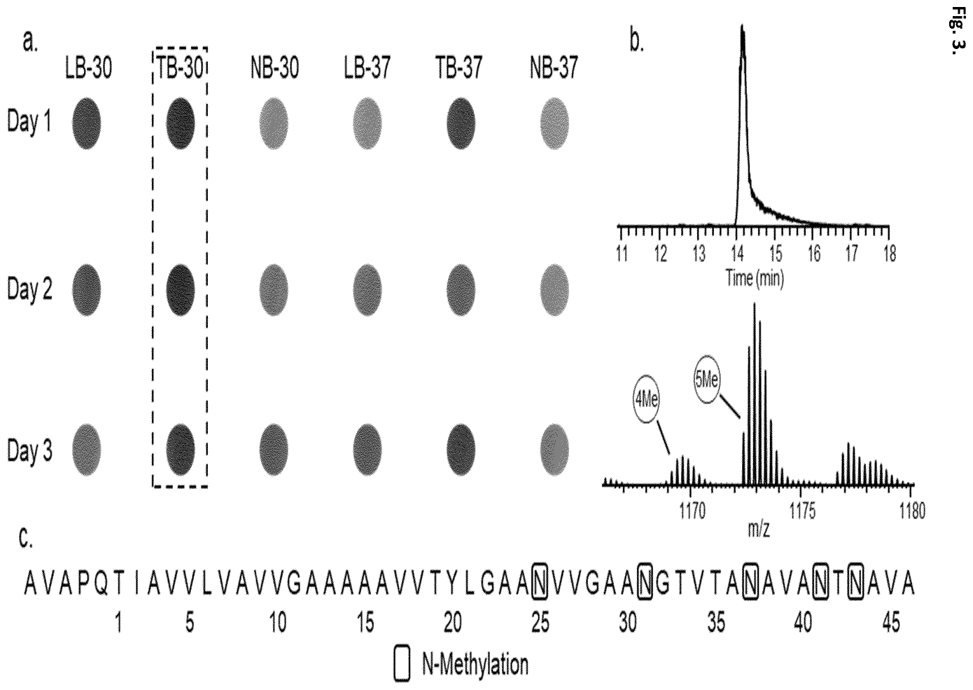

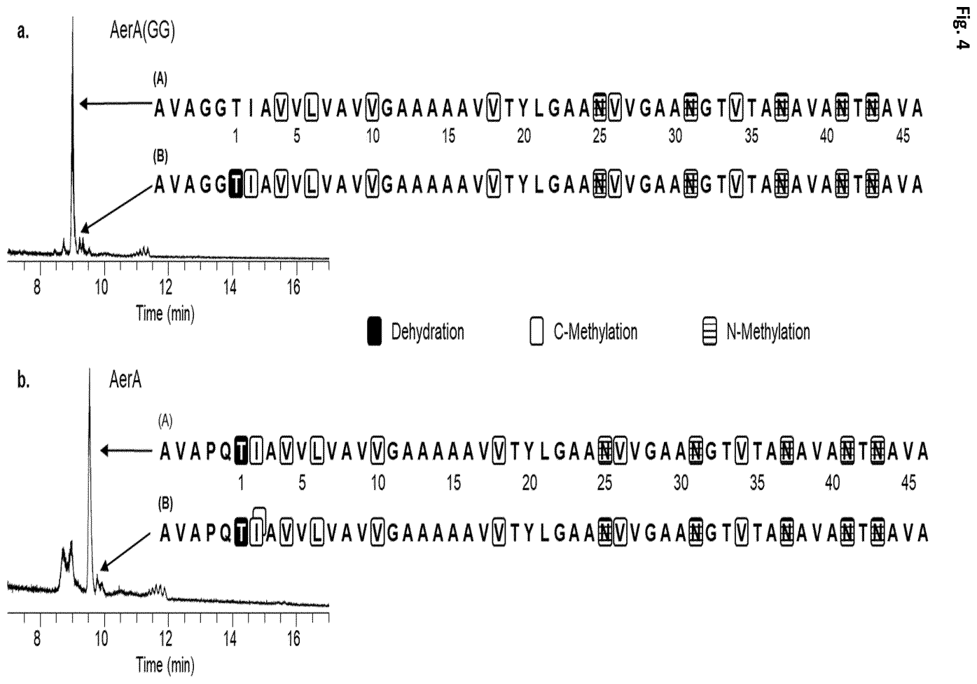

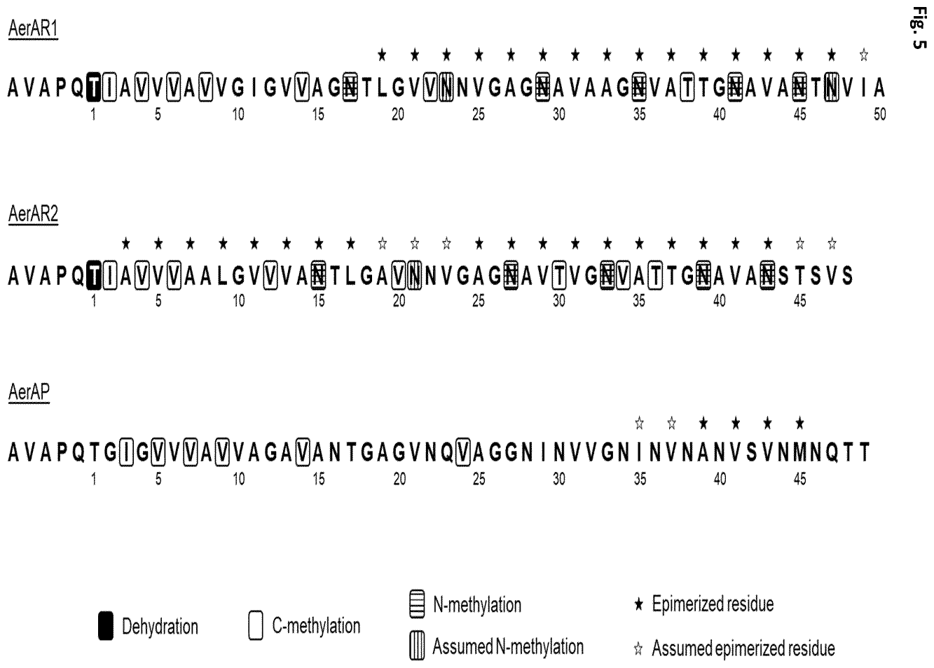

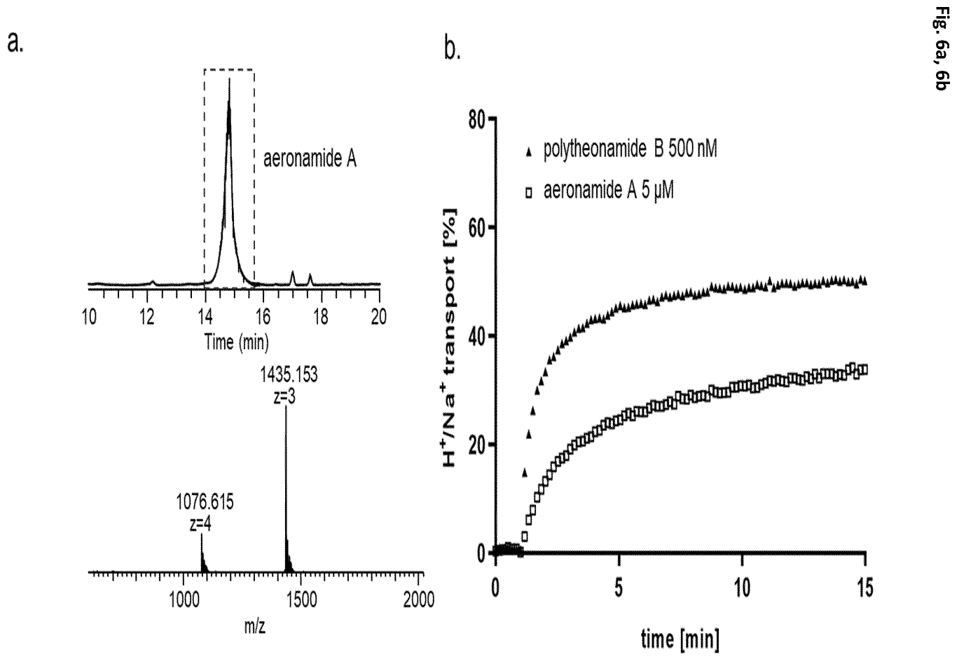

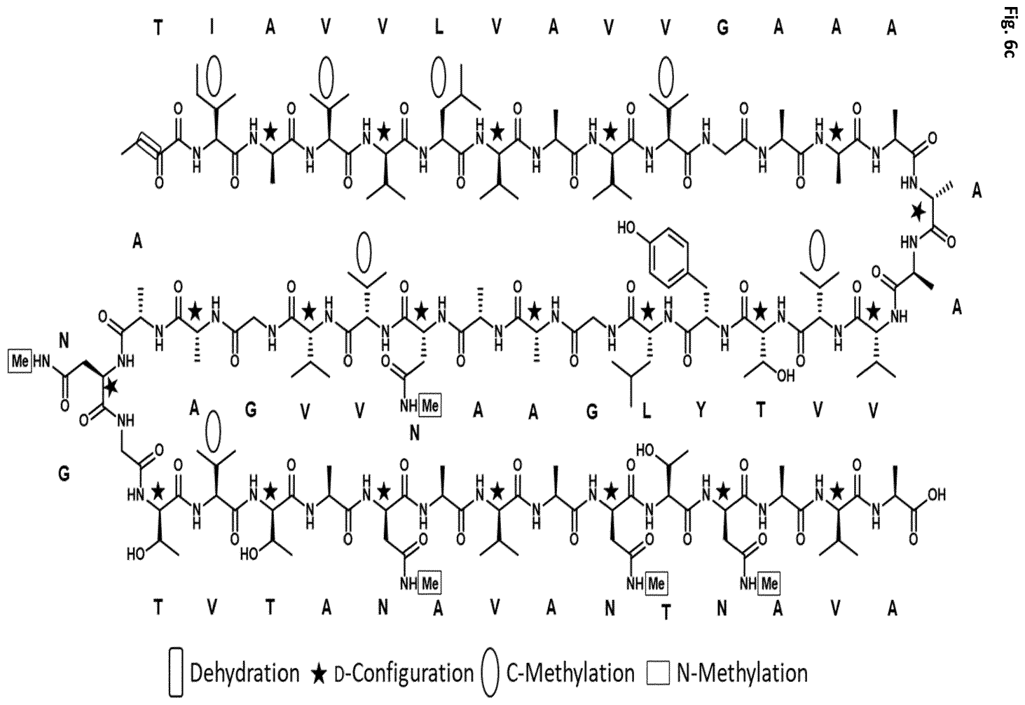

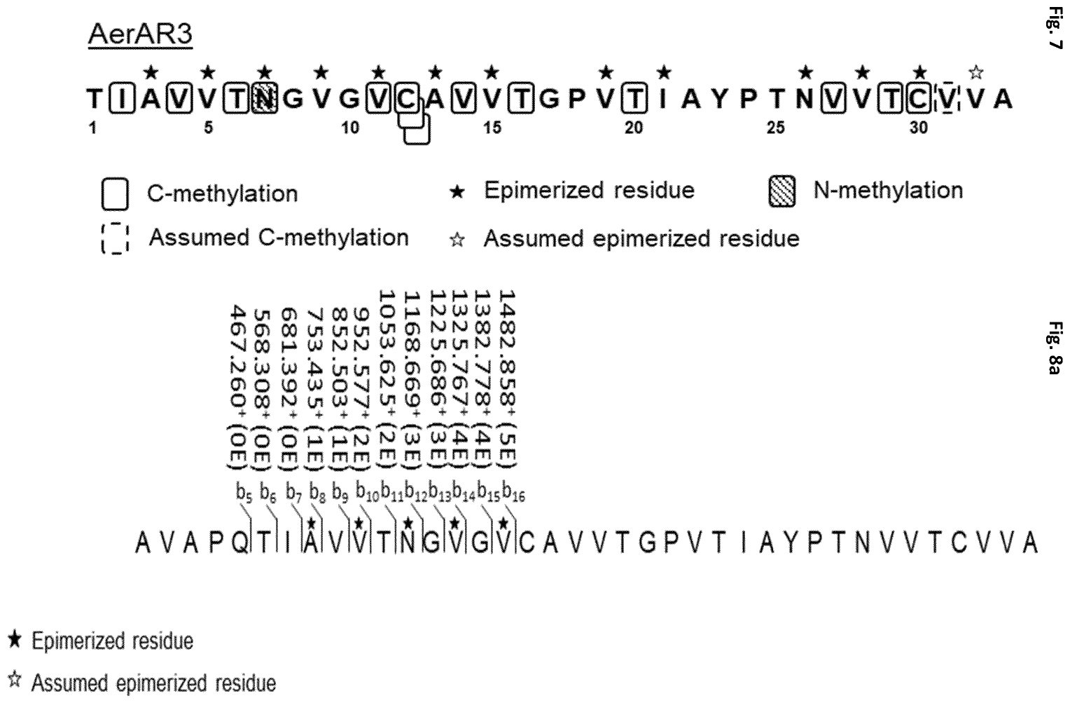

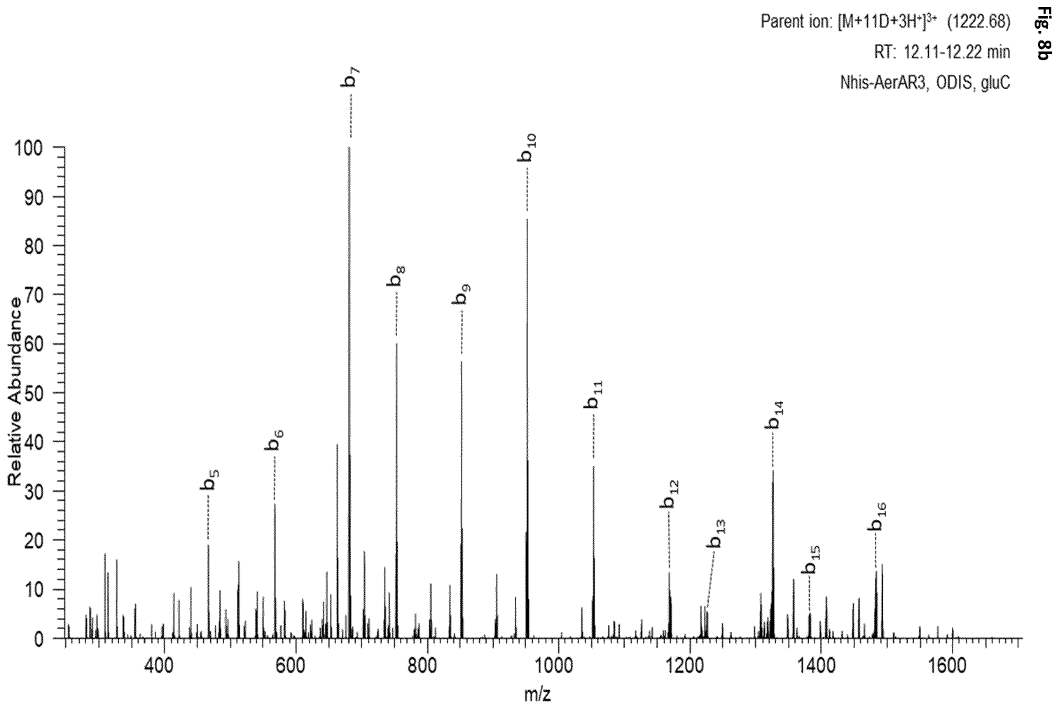

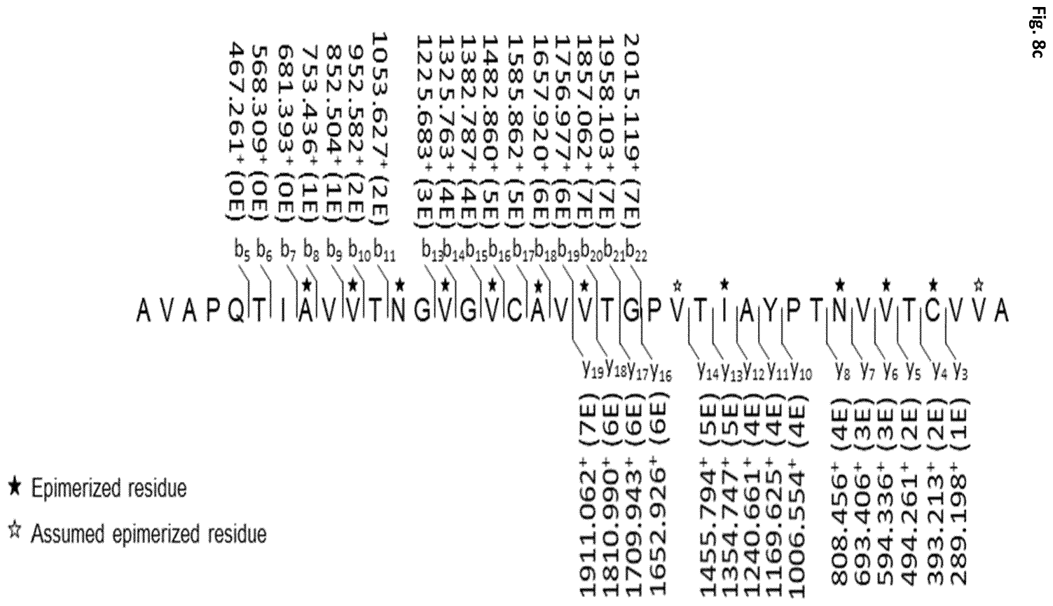

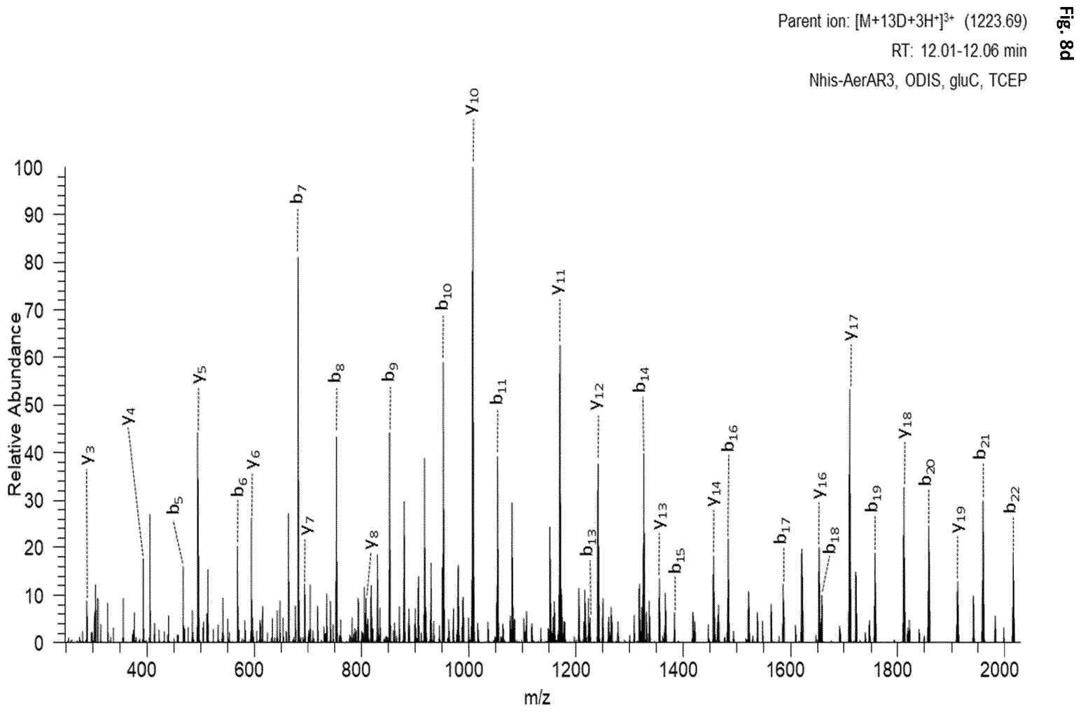

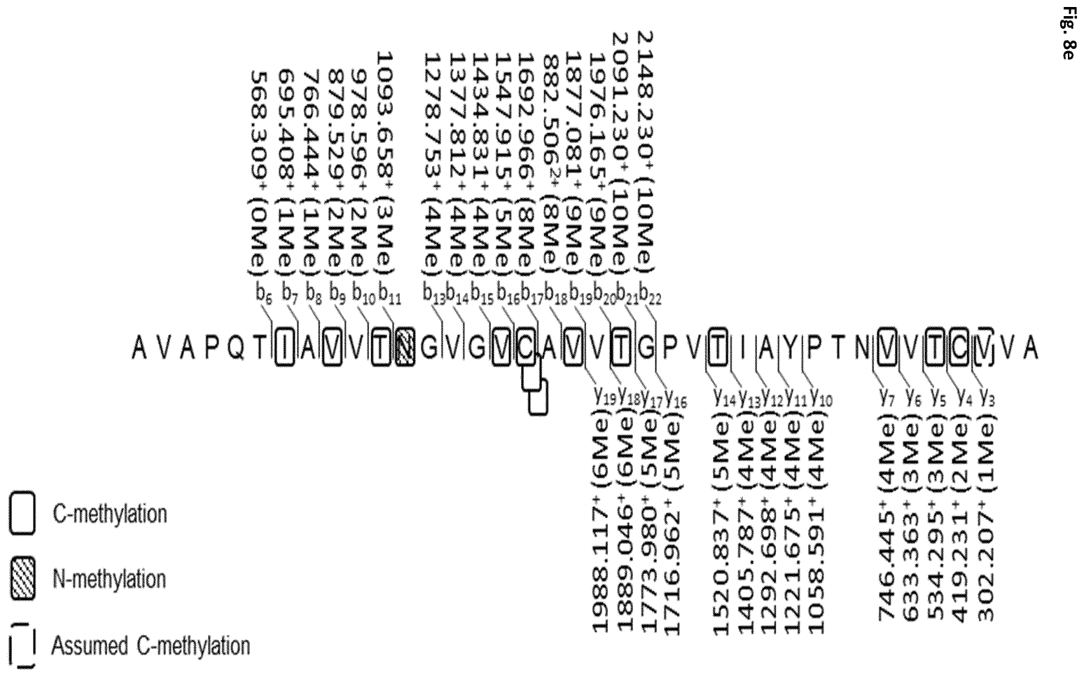

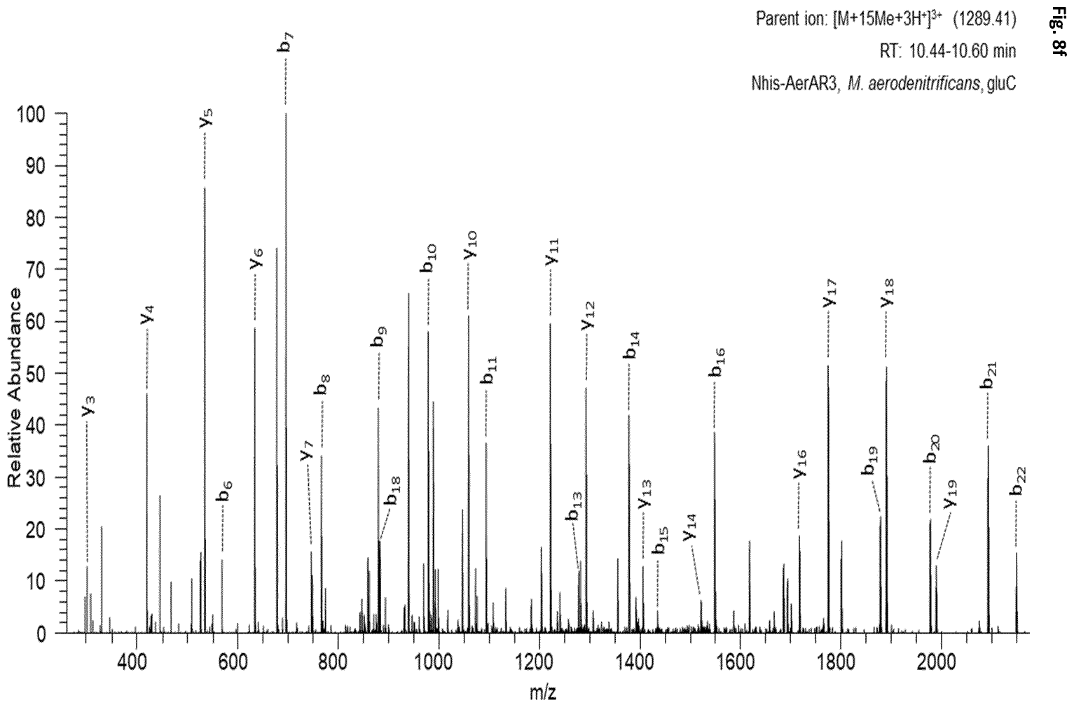

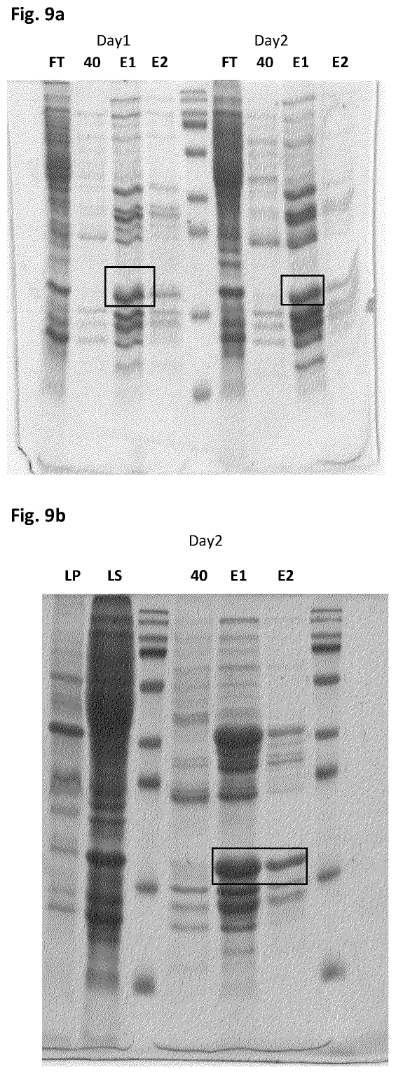

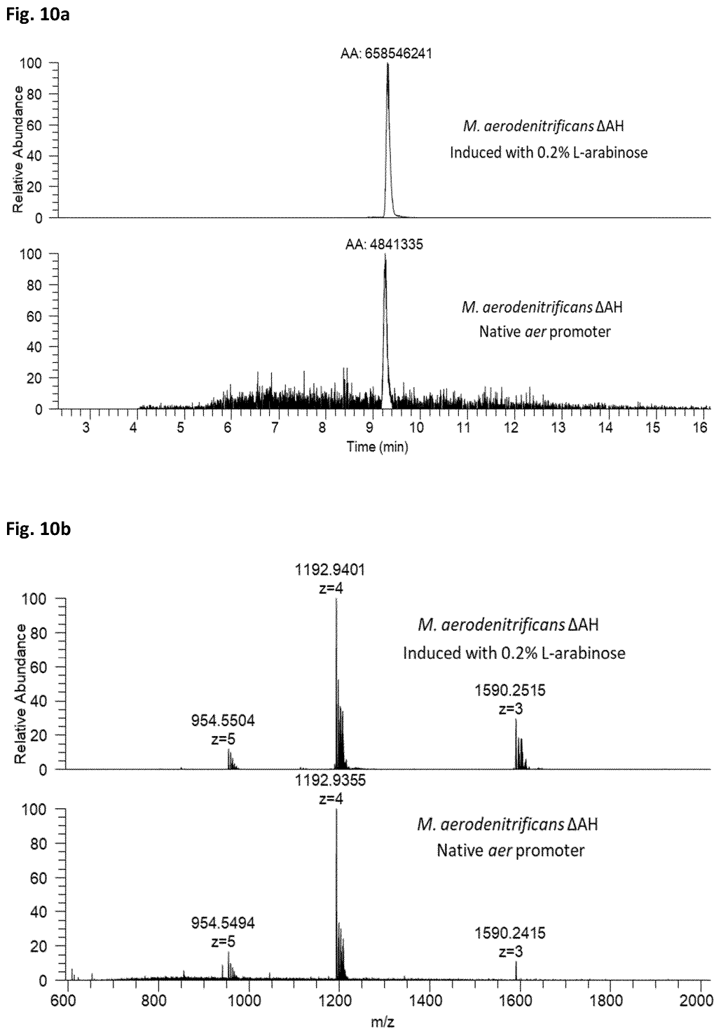

SEQ ID NO: 12, see above FIG. 1C

FIG. 5. Modifications localized to other cores expressed in M. aerodenitrificans using the tagged-bait strategy. Epimerizations localized via ODIS expressions in E. coli. Assumed modifications refer to modifications that could not be localized to a particular residue during MS² analysis, but for which fragmentation supported the existence of such a modification.

AerAR1 a.a. Microvirgula aerodenitrificans
(SEQ ID NO: 19)
AVAPQTIAVVVAVVGIGVVAGNTLGVVNNVGAGNAVAAGNVATTGNAVAN
TNVIA
AerAR2 a.a. Microvirgula aerodenitrificans
(SEQ ID NO: 20)
AVAPQTIAVVVAALGVVVANTLGAVNNVGAGNAVTVGNVATTGNAVANST
SVS
AerAP a.a. Microvirgula aerodenitrificans
(SEQ ID NO: 21)
AVAPQTGIGVVVAVVAGAVANTGAGVNQVAGGNINVVGNINVNANVSVNM
NQTT

FIGS. 6a, 6b, and 6c. (6a) TIC (left) and mass spectra (right) of HPLC purified aeronamide A following in vitro cleavage of Nhis-AerA (from M. aerodenitrificans) with Nhis-AerH (from E. coli). (6b) Results of H⁺/Na⁺ ion exchange activity assay on artificial liposomes for aeronamide A and polytheonamide B. (6c) Structure of aeronamide A, SEQ ID NO: 12, see above FIG. 1C. Modified residues indicated according to the legend below. The orange balloons above the residues point to the residue a methylation was localized to, but without knowledge of the specific position of modification on the side chains.

FIG. 7. Modifications localized to AerAR3 expressed in M. aerodenitrificans using the tagged-bait strategy. Epimerizations localized via ODIS expressions in E. coli. Assumed modifications refer to modifications that could not be localized to a particular residue during MS² analysis, but for which fragmentation supported the existence of such a modification.

AerAR3 a.a. Microvirgula aerodenitrificans
(SEQ ID NO: 22)
TIAVVTNGVGVCAVVTGPVTIAYPTNVVTCVVA
AerAR3 nucleotide sequence
Microvirgula aerodenitrificans
(SEQ ID NO: 23)
ACCATCGCCGTCGTCACCAACGGCGTCGGCGTGTGCGCAGTCGTGACCGG
CCCGGTGACCATCGCCTATCCCACGAACGTGGTGACTTGCGTCGTCGCCT
GA

FIGS. 8a, 8b, 8c, 8d, 8e, and 8f. MSMS fragmentation masses of AerAR3 observed are listed above (b-ions) and below (y-ions) the denoted sequence ((8a), (8c), (8e)) with black lines marking the site of fragmentation. For each MSMS spectrum ((b), (d), (f)), the parent ion information, HPLC retention time (RT), the shorthand notation for the expression, and the protease used post purification is listed in the upper right-hand corner of the spectrum. Ions observed to the corresponding peak in the spectra are marked by a dotted line. LC method 1 (see below) ((8a), (8b), (8c), (8d)) and LC method 3 (see below) ((8e), (8f)) with PRM mediated MSMS fragmentation. Masses of PTM-containing ions are denoted in brackets, where ‘Me’ denotes a mass shift corresponding to a methylation and E to an epimerization (incorporation of a deuterium). The residues localized to the PTM are marked according to legend (top left of spectra). (8a), (8b): Nhis-AR3 ODIS; (8c), (8d): Nhis-AerAR3 ODIS treated with TCEP; (8e), (8f): Nhis-AerAR3. ODIS expressions ((8a), (8b), (8c) and (8d)) were carried out in E. coli.

Nhis-AerAR3 a.a.
Microvirgula aerodenitrificans
(SEQ ID NO: 24)
AVAPQTIAVVTNGVGVCAVVTGPVTIAYPTNVVTCVVA

FIGS. 9a and 9b. 15% SDS-PAGE (stained with Coomassie Brilliant Blue) of Nhis-AerA precursor expressed in M. aerodenitrificans ΔAH (a) and M. aerodenitrificans (b) under the control of the arabinose promoter. The square boxes highlight the bands of Nhis-AerA. Expression was induced with 0.2% w/v L-arabinose. Abbreviations: LP—lysis pellet; LS—lysis supernatant; FT—flow through; 40-40 mM imidazole wash; E1—first 250 mM imidazole elution; E2—second 250 mM imidazole elution.

FIGS. 10a and 10b. (10a) Extracted ion chromatogram and (10b) corresponding spectrum from LC-MS analysis of Nhis-AerA expressed under the Pimp arabinose promoter (top in (10a) and (10b)) and under the native aer promoter (bottom in (10a) and (10b)). AA value represents area under the peak.

### DETAILED DESCRIPTION OF THE INVENTION

### Examples

Materials

Restriction enzymes, Q5 site-directed mutagenesis kit, and Gibson assembly mixtures were purchased from New England Biolabs. Thermo Scientific Phusion® DNA polymerase and T4 DNA ligase were used for all PCR reactions and ligations, respectively. PCR primers were supplied by Microsynth and are listed in the ‘Oligonucleotides’ column of Table S2. Commercial proteases were purchased from Applichem (proteinase K) and New England Biolabs (Endoproteinase GluC). Solvents for HPLC-MS analyses were Optima® LC-MS grade from Fisher Scientific and HPLC grade from Acros Organics and Sigma-Aldrich. Unless otherwise stated, chemicals were purchased from Sigma-Aldrich.

For all HPLC-MS analysis a Phenomenex Kinetex 2.6 μm C18 100 Å (150×4.6 mm) was used on a Dionex Ultimate 3000 UHPLC system coupled to a Thermo Scientific Q Exactive mass spectrometer. Unless otherwise stated, the columns were heated to 50° C. For expression products derived from E. coli and AerAP expressions in M. aerodenitrificans, the solvents used were water with 0.1% (v/v) formic acid (solvent A) and acetonitrile with 0.1% (v/v) formic acid (solvent B). A general LC method was used in this case; LC method 1: at a flow rate of 0.5 mL/min, solvent B was 5% from 0 to 2 min, 5% to 98% from 2 to 15 min, 98% from 15 to 20 min, 98% to 5% from 20 to 22 min, and 5% from 22 to 24.5 min. For all other expressions in M. aerodenitrificans, the solvents used were water with 0.5% (v/v) formic acid (or 0.1% TFA) as solvent A and n-propanol 0.5% (v/v) formic acid (or 0.1% TFA) as solvent B. Two different methods were used with LC method 2: at a flow rate of 0.75 mL/min, solvent B was 25% from 0 to 2 min, 25% to 65% from 2 to 20 min, 98% from 20.5 to 30 min, 98% to 25% from 30 to 32 min, and 25% from 32 to 32.5 min. LC method 3: at a flow rate of 0.75 mL/min, solvent B was 25% from 0 to 2 min, 25% to 65% from 2 to 30 min, 98% from 30.5 to 40 min, 98% to 25% from 40 to 42 min, and 25% from 42 to 42.5 min. The corresponding methods used for each sample or batches of runs are noted in their respective sections. Unless otherwise stated, ESI-MS was performed in positive ion mode, with a spray voltage of 3500 V, a capillary temperature of 268.75° C., probe heater temperature ranging from 350° C. to 437.5° C. and an S-lens level range between 50 and 70. Full MS was done at a resolution of 35,000 (AGC target 2e5, maximum IT 100 ms, range 600-2000 m/z). Parallel reaction monitoring (PRM) or data-dependent MSMS was performed at a resolution of 17500 (AGC target between 1e5 and 1e6, maximum IT between 100 ms and 250 ms, isolation windows in the range of 1.1 to 2.2 m/z) using a stepped NCE of 18, 20 and 22 or an NCE of 18. Scan ranges, inclusion lists, charge exclusions, and dynamic exclusions were adjusted as needed.

### Example 1—Microvirgula aerodenitrificans Transformation

M. aerodenitrificans DSMZ 15089 primary cultures containing 20 mL nutrient broth (NB) medium (5.0 g peptone, 3.0 g meat extract per 1.0 L) were inoculated from a glycerol stock and grown in a shaker to saturation for 1 day at 180 rpm and 30° C. E. coli SM10 strains harboring various plasmids were grown overnight to saturation in 20 mL LB at 250 rpm and 37° C. Both strains were harvested by centrifugation (10,000×g), washed with a 0.9% (w/v) NaCl solution, and resuspended in 0.9% (w/v) NaCl solution that was then adjusted to an OD₆₀₀ of 4.0. Ratios of donor (SM10) and recipient (M. aerodenitrificans) strains of 1:9, 3:7, and 1:1 (v/v) were prepared and vortexed in a 1.0 mL final volume, spun down at 16,000×g for 1 min, and resuspended in 50 μl 0.9% (w/v) NaCl solution. Cell mixtures were spotted on nutrient agar plates (1.5% (w/v) agar) and let dry prior to incubation at 37° C. for two days. The resulting mixed-cellular growths of different ratios were then removed from the plate with a sterile loop and transferred into 1.0 mL of a 0.9% (w/v) NaCl solution. Cell solutions (100 μL) were then plated out on selective NA plates containing gentamycin (10 μg/mL final concentration; positive selection for the pLMB509 plasmid) and carbenicillin (400 μg/mL final concentration; negative selection for SM10). Plates were incubated at 30° C. for up to 2 days.

### Example 2—Culturing Conditions

M. aerodenitrificans: Starter cultures (20 mL NB with 10 μg/mL gentamycin) were inoculated from a glycerol stock or a fresh colony harboring pLMB509-derived plasmids and grown overnight at 30° C. and 180 rpm. 200 μL of the culture was used to inoculate freshly prepared Terrific Broth (TB) media (20 mL with 10 μg/mL gentamycin) and grown overnight. 4 mL of the cultures was then used to inoculate 400 mL of TB media in 2 L Erlenmeyer flasks, grown at 30° C. and 180 rpm for 1-4 days. The cells were harvested via centrifugation, flash frozen in liquid nitrogen and stored at −80° C. until use.

E. coli: Plasmids were transformed in BL21 Star (DE3) unless otherwise stated and expression cultures were inoculated from overnight cultures in a 1:100 (v % v) dilution in 1 LTB medium. Cells were grown at 37° C., 250 rpm to OD₆₀₀ 1.6-2 in 2.5 L Ultra Yield Flasks (Thompson). Flasks were then chilled in an ice bath for 30 min followed by addition of 1 mM IPTG (final concentration) and incubation at 16° C., 250 rpm for 18 hours, unless otherwise stated.

### Example 3—Protein Purification

For all AerA variants, the same lysis method was used: Cells were resuspended in lysis buffer (20 mM imidazole, 50 mM sodium phosphate pH 8.0, 300 mM NaCl, 10% (v/v) glycerol) supplemented with 0.01% (v/v) Triton X-100 and 1 mg/mL lysozyme (Carl Roth) (final concentrations) in a ratio of 1 g wet cell weight to 4 mL lysis buffer. Cell suspensions were incubated at 37° C. and 250 rpm for 30 min and sonicated using a Qsonica Q700 sonicator with a 6 mm probe for 15 cycles of 10 s pulse/10 s rest at 25% amplitude followed by centrifugation at 18,000×g (4° C., 30 min). The resulting supernatant was incubated with 0.5-1 mL Protino Ni-NTA resin (Macherey-Nagel) for 1 h at 4° C. with gentle rocking. The Ni-NTA resin was then pelleted at 800×g for 15 min, transferred to a fritted column, and washed with 1 round of 15 mL lysis buffer prior to protein elution with 2 rounds of 0.5-1.0 mL elution buffer (250 mM imidazole, 50 mM sodium phosphate pH 8.0, 300 mM NaCl, 10% (v/v) glycerol). When required, the elution fraction was concentrated sufficiently with Amicon Ultra centrifugal filters (3k or 5k MWCO, Millipore).

### Example 4—Orthogonal D₂O-Based Induction System (ODIS) for Labeling Epimerized Core Peptides

Nhis-precursor peptides in pACYCDuet-1 was cotransformed with the AerD gene in pCDFBAD/Myc-His A (pBAD/Myc-His A vector with the native origin of replication replaced by that of pCDFDuet) in E. coli BL21 (DE3) cells and plated on LB agar containing chloramphenicol (25 μg/mL) and ampicillin (100 μg/mL) and grown for 20 h at 37° C. or until colonies appeared. These colonies were used to inoculate 20 mL LB with chloramphenicol (25 μg/mL) and ampicillin (100 μg/mL) and grown overnight. The following day, nine separate 50 mL falcon tubes containing TB media (15 mL), chloramphenicol (25 μg/mL) and ampicillin (100 μg/mL) were inoculated with 150 μL and shaken at 37° C., 250 rpm to OD₆₀₀ 1.6-2. Cultures were cooled on ice for 30 minutes, induced with IPTG (0.1 mM final concentration), and shaken (200 rpm, 16° C.) for 16 hours. The cultures were centrifuged (20 minutes, 10,000×g) and the supernatant removed. The cell pellets were then washed with TB medium (2×15 mL) to remove any residual IPTG. In the second wash, the cells were shaken (200 rpm, 16° C.) for 1 hour to further metabolize intracellular IPTG. The washed cell pellets were resuspended in 15 mL TB medium in D₂O containing ampicillin (100 μg/mL in D₂O), and L-arabinose (100 L, 20% w/v in D₂O) and shaken (200 rpm, 16° C.) for 18 hours. The cultures were combined and centrifuged (30 minutes, 15,000×g) and the pellet resuspended in 10 mL lysis buffer and treated as described in example 4.

### Example 5—Proteolytic Cleavage for Analysis of Core Peptides and Generation of the Core Region

GluC cleavage: To analyse the post-translational modifications on the core peptide, between 20-40 μL of the elution fraction was mixed with 50 μL 2×GluC buffer and 10 μL GluC (0.25 μg/mL) to have a final volume of 100 μL and incubated at 37° C. for 16 hrs before analysis by LC-MS.

Proteinase K digest: 16 μL of the elution was mixed with 20 μL of proteinase K buffer (100 mM Tris, 4 mM CaCl₂, pH 8.0) 4 μl of proteinase K (2 mg/mL). For the elutions arising from expression in E. coli, this reaction was carried out in PCR tubes (12 h, 50° C.), while for elutions from expression in M. aerodenitrificans was carried out in glass inlets (12 h, 37° C.).

AerH digest: For small-scale reactions, typically 13 μL of the peptide elutions were mixed with 7 μl of Nhis-AerH (23 mg/ml) and 20 μL of proteinase K buffer. For large scale reactions, 2.4 mL of the peptide elution was mixed with 200 μL of Nhis-AerH and 2.6 mL of proteinase K buffer. All reactions were done in glass vials. The reaction was then spun down in glass tubes (2,000×g, 20 min) with the supernatant collected and the pellet being redissolved in 2 mL propanol. This was again centrifuged (2,000×g, 20 min) and the supernatant collected.

### Example 6—Glucuronidase Activity Assay

Culture volumes equaling an OD₆₀₀ of 20 were centrifuged (10,000×g, 10 mins) and the pellets resuspended in 1 mL lysis buffer (50 mM phosphate buffer pH 7.0, 5 mM dithiothreitol, 0.1% Triton X-100, 1 mg/ml lysozyme). Lysis was performed at 37° C. for 15 min followed by sonication using a Qsonica Q700 sonicator and 4420 microtip for 10 cycles of 10 s pulse/10 s rest at 25% amplitude. Lysates were centrifuged at 10,000×g for 10 min. Then, 0.5 ml of lysate was supplemented with 10 μL 10 mg/mL X-glucuronide (5-Bromo-4-chloro-3-indolyl β-D-glucuronide) and incubated for 1 hour at 37° C.

### Example 7—Preparation of Pyranine-Encapsulated LUV's

To create large unilamellar vesicles (LUVs) a solution of 27.5 mg 1,2-Dimyristoyl-sn-glycero-3-phosphorylcholine (DMPC) and 8 mg cholesterol in CHCl₃ was dried to completeness under vacuum to form a thin lipid layer. The thin layer was suspended in 2 ml of trisodium 8-hydroxypyrene-1,3,6-trisulfonate (pyranine)-containing buffer (15 mM Hepes, pH 6.5, 200 mM NaCl, 1 mM pyridine) by mild sonication under argon gas. After five-times freeze-thaw cycles in liquid nitrogen, the lipid suspension was extruded 30 times through a polycarbonate filter with a pore size of 0.2 μm using the Avanti Mini Extruder (Avanti Polar Lipids, Alabaster, Ala., USA). Residual external pyranine dye was subsequently removed by size exclusion chromatography using a PD-10 desalting column. The resulting solution was adjusted to 1 mM with dye free resuspension buffer (15 mM Hepes pH 6.5, 200 mM NaCl). For the H+/Na+ exchange assay the liposome solution was diluted to 50 μM with assay buffer (15 mM HEPES pH 7.5, 200 mM NaCl) to create a pH gradient.

### Example 8—H⁺/Na⁺ Exchange Assay

A suspension of pyranine-loaded LUV's was placed into a quartz cuvette (2 ml). The fluorescence emission was measured at 511 nm with an excitation at 460 nm in a Varian Cary Eclipse spectrofluorimeter. After 60s, peptides in DMSO were added at indicated concentrations and the fluorescence emission was recorded for 15 min at a sampling rate of 0.1 s. Afterwards LUVs were completely lysed by the addition of 5 μl of a 10% Triton X-100 aqueous solution. The background drift by the addition of pure DMSO was subtracted from all traces. The data was normalized against 100% lysis by Triton X-100.

### Example 9—Cell-Free Assay

Wild-type M. aerodenitrificans was grown in 200 mL TB media at 30° C. for one day. 30 mL of the culture was centrifuged at 18,000×g for 30 minutes and the cell pellet was resuspended in 1 mL ammonium acetate buffer (50 mM ammonium acetate, 10% v/v glycerol and 50 mM potassium chloride, pH 5). The cells were then lysed using Qsonica Q700 sonicator and 4420 microtip for 10 cycles of 10 s pulse/10 s rest at 25% amplitude. Lysates were centrifuged at 11,000×g for 30 min and the supernatant collected. To 1 mL of the lysate supernatant, 100 μL of Nhis-AerAD from E. coli was added and incubated for 2 days followed by affinity purification as described above. After purification, the sample was treated by gluC and analysed by LC-MS.

### Example 10—Cytotoxic Assays

The activity of aeronamide A was measured against HeLa cells. Stocked HeLa cells were resuspended in 10 mL HEPES buffered high glucose Dulbecco's Modified Eagle Medium (DMEM) supplemented with GlutaMAX (Gibco). Additionally, the medium contained 10% fetal calf serum (FCS) and 50 mg/mL gentamycin. The cells were centrifuged for 5 min at 1000×g and room temperature. The medium was discarded and the cells resuspended in 10 mL fresh medium. The cells were put in a culture dish and incubated for 3-4 days at 37° C. The cells were checked under the microscope and treated further only when 60-80% of the surface was covered with cells. The medium was removed from the culture flask and the cells were washed with 10 mL phosphate buffered saline (PBS). The PBS was discarded and the cells treated with 2 mL Trypsin-EDTA solution. When the cells were detached, 10 mL of medium was added and centrifuged for 5 min at 1000×g and room temperature. The supernatant was discarded and 10 mL fresh medium were added. 2 mL of the cell suspension were put in a fresh culture flask containing 10 mL medium. Cells healthy enough for cytotoxicity assays were counted and diluted to a 10,000 cells/mL solution. 96 well plates were filled with 200 μL cell suspension per well. All plates were incubated overnight at 37° C. The outer wells were not used for the assay. 2 μL of test solutions in DMSO were put in the B lane wells. Aeronamide A was a 1 mM solution, doxorubicin was used as a positive control at 1 mg/mL, and DMSO was used as negative control. 50 μL of lane B were transferred into lane C and mixed, and this transfer to the adjacent lane was repeated until lane G. The plates were then incubated for 3 days. 50 μL of 3-(4,5-dimethylthiazol-2-yl)-2,5-diphenyltetrazolium bromide (MU) (1 mg/mL in water) were added to all wells and incubated for 3 h at 37° C. The supernatant was discarded and 150 μL of dimethyl sulfoxide (DMSO) were added to all wells. Absorbance was measured at 570 nm and IC₅₀ was calculated using GraphPad Prism 6 (GraphPad).

### Example 11—HPLC-MS Analysis

For all HPLC-MS analysis a Phenomenex Kinetex 2.6 μm C18 100 Å (150×4.6 mm) was used on a Dionex Ultimate 3000 UHPLC system coupled to a Thermo Scientific Q Exactive mass spectrometer. Unless otherwise stated, the columns were heated to 50° C. For expression products derived from E. coli and AerAP expressions in M. aerodenitrificans, the solvents used were water with 0.1% (v/v) formic acid (solvent A) and acetonitrile with 0.1% (v/v) formic acid (solvent B). A general LC method was used in this case; LC method 1: at a flow rate of 0.5 mL/min, solvent B was 5% from 0 to 2 min, 5% to 98% from 2 to 15 min, 98% from 15 to 20 min, 98% to 5% from 20 to 22 min, and 5% from 22 to 24.5 min. For all other expressions in M. aerodenitrificans, the solvents used were water with 0.5% (v/v) formic acid (or 0.1% TFA) as solvent A and n-propanol 0.5% (v/v) formic acid (or 0.1% TFA) as solvent B. Two different methods were used with LC method 2: at a flow rate of 0.75 mL/min, solvent B was 25% from 0 to 2 min, 25% to 65% from 2 to 20 min, 98% from 20.5 to 30 min, 98% to 25% from 30 to 32 min, and 25% from 32 to 32.5 min. LC method 3: at a flow rate of 0.75 mL/min, solvent B was 25% from 0 to 2 min, 25% to 65% from 2 to 30 min, 98% from 30.5 to 40 min, 98% to 25% from 40 to 42 min, and 25% from 42 to 42.5 min. The corresponding methods used for each sample or batches of runs are noted in their respective sections. Unless otherwise stated, ESI-MS was performed in positive ion mode, with a spray voltage of 3500 V, a capillary temperature of 268.75° C., probe heater temperature ranging from 350° C. to 437.5° C. and an S-lens level range between 50 and 70. Full MS was done at a resolution of 35,000 (AGC target 2e5, maximum IT 100 ms, range 600-2000 m/z). Parallel reaction monitoring (PRM) or data-dependent MSMS was performed at a resolution of 17500 (AGC target between 1e5 and 1e6, maximum IT between 100 ms and 250 ms, isolation windows in the range of 1.1 to 2.2 m/z) using a stepped NCE of 18, 20 and 22 or an NCE of 18. Scan ranges, inclusion lists, charge exclusions, and dynamic exclusions were adjusted as needed.

### Example 12—Purification of Aeronamide

Supernatants from the AerH digest were combined and diluted to 5% propanol and passed through a Phenomenex Strata® C18-E (55 μm, 70 Å) 5 g/20 mL column. The column was then washed with 4 column volumes of Milli Q water followed by 1 column volume of acetonitrile. Aeronamides were then eluted with 3 column volumes of n-propanol and evaporated using GeneVac EZ-2 Elite. The resulting pellet was dissolved in 75% propanol and separated by RP-HPLC (Phenomenex Luna 5p. C18, 10×250 mm, 2.4 mL/min, 200 nm) with a gradient elution from 25% n-propanol to 65% n-propanol from 2 to 30 min, with fractions collected and analyzed by LC-MS. Aeronamide A eluted between 26.5-27.5 min.

### Example 13—P_{BAD} Arabinose Promoter

Using Gibson assembly, the PBAD arabinose promoter derived from plasmid psw8197 (see F. Le Roux et al. 2007, Applied and Environmental Microbiology, 777-784) was inserted in place of the aer promoter in the plasmid p509, with a 13 bp ribosomal binding site of the aer promoter remaining in place before the Nhis-aerA gene to be expressed. The plasmid was conjugated in to wild-type and mutant (ΔAH) M. aerodenitrificans, with a single colony picked for growth and expression. The promoter sequence (SEQ ID NO: 25) is shown below and the functional elements are highlighted as follows: Bold: Arabinose regulator, AraC; Italic: Arabinose promoter sequence; Normal: aer promoter ribosomal binding site (RBS)

TTATGACAACTTGACGGCTACATCATTCACTTTTTCTTCACAACCGGCAC
GAAACTCGCTCGGGCTGGCCCCGGTGCATTTTTTAAATACTCGCGAGAAA
TAGAGTTGATCGTCAAAACCAACATTGCGACCGACGGTGGCGATAGGCAT
CCGGGTAGTGCTCAAAAGCAGCTTCGCCTGACTAATGCGTTGGTCCTCGC
GCCAGCTTAAGACGCTAATCCCTAACTGCTGGCGGAAAAGATGTGACAGA
CGCGACGGCGACAAGCAAACATGCTGTGCGACGCTGGCGATATCAAAATT
GCTGTCTGCCAGGTGATCGCTGATGTACTGACAAGCCTCGCGTACCCGAT
TATCCATCGGTGGATGGAGCGACTCGTTAATCGCTTCCATGCGCCGCAGT
AACAATTGCTCAAGCAGATTTATCGCCAGCAGCTCCGAATAGCGCCCTTC
CCCTTGCCCGGCGTTAATGATTTGCCCAAACAGGTCGCTGAAATGCGGCT
GGTGCGCTTCATCCGGGCGAAAGAAACCCGTATTGGCAAATATTGACGGC
CAGTTAAGCCATTCATGCCAGTAGGCGCGCGGACGAAAGTAAACCCACTG
GTGATACCATTCGCGAGCCTCCGGATGACGACCGTAGTGATGAATCTCTC
CTGGCGGGAACAGCAAAATATCACCCGGTCGGCAGACAAATTCTCGTCCC
TGATTTTTCACCACCCCCTGACCGCGAATGGTGAGATTGAGAATATAACC
TTTCATTCCCAGCGGTCGGTCGATAAAAAAATCGAGATAACCGTTGGCCT
CAATCGGCGTTAAACCCGCCACCAGATGGGCGTTAAACGAGTATCCCGGC
AGCAGGGGATCATTTTGCGCTTCAGCCATACTTTTCATACTCCCACCATT
CAGAGAAGAAACCAATTGTCCATATTGCATCAGACATTGCCGTCACTGCG
TCTTTTACTGGCTCTTCTCGCTAACCCAACCGGTAACCCCGCTTATTAAA
AGCATTCTGTAACAAAGCGGGACCAAAGCCATGACAAAAACGCGTAACAA
AAGTGTCTATAATCACGGCAGAAAAGTCCACATTGATTATTTGCACGGCG
TCACACTTTGCTATGCCATAGCATTTTTATCCATAAGATTAGCGGATCCT
ACCTGACGCTTTTTATCGCAACTCTCTACTGTTTCTCCATACCCGTTTTT
TTAGGAGAGTGCGGG

Expression: An overnight culture of Microvirgula (WT and Knockouts) grown in nutrient broth was used to inoculate 20 mL of TB media (with gentamycin 10 μg/mL) and grown at 30° C. overnight. 4 mL of this culture was used to inoculate 400 mL of TB media (with gentamycin 10 μg/mL), which was subsequently grown to an OD₆₀₀ of 0.6, induced with 0.2% w/v of L-Arabinose and grown over a period of two days. The cells were collected by centrifugation, lysed and Ni-affinity purified (see FIG. 9). The purified protein was treated with GluC and analysed by LC-MS, with a control sample of Nhis-AerA expressed under the aer promoter (see FIG. 10). The yield observed for the GluC generated aeronamide A was more than a 100-fold, when expressed under the new PBAD promoter.

### Summary of the Examples

pLMB509 was first developed as regulatable expression vector for use in Alphaproteobacteria (Appl Environ Microbiol 2012, 78(19): 7137-7140). The vector is derived from pRU1097 (pBBR origin of replication) and has an origin of transfer enabling conjugation and gentamycin resistance. For protein expression, a taurine inducible promoter system is present with a downstream gfpmut3.1 reporter gene. To test for expression of the aer cluster in M. aerodenitrificans, the vector pLMB509 was modified by replacing the taurine induction system and gfpmut3.1 with the aer promoter (362 bp upstream region from aerC) proceeded by the reporter gene gusA, encoding the enzyme glucuronidase A (example 6). This modified vector was transformed into M. aerodenitrificans and grown under different conditions. These conditions included LB—luria bertani medium, TB—terrific broth, NB—nutrient broth, MB—marine broth and temperatures of 30° C. and 37° C., with samples being collected at day 1, day 2 and day 3 and frozen. The frozen samples were lysed, centrifuged and the supernatant was incubated with X-glucuronide (5-Bromo-4-chloro-3-indolyl β-D-glucuronide) for hour. Of the conditions tested, cultivation of the M. aerodenitrificans reporter strain over a period of three days at 30° C. in terrific broth (TB) medium, routinely used for protein expression in E. coli, resulted in strong induction of GusA activity already after one day (FIG. 3a). The activity of the aer cluster was further established by incubating Nhis-AerAD from E. coli with cell free lysate from M. aerodenitrificans as described in example 9 resulting in the methylation of all 5 asparagine residues (FIG. 3b, c).

The modified pLMB509 vector, with Nhis-AerAX under the control of the aer promoter was successfully transformed into Microvirgula aerodenitrificans as described in example 1. Nhis-AerX includes Nhis-AerA, Nhis-AerAR1, Nhis-AerAR2, Nhis-AerAR3, Nhis-AerAP, Nhis-AerA(GG), Nhis-AerAR1(GG), Nhis-AerAR2(GG), Nhis-AerAR3(GG), Nhis-AerAP(GG) and Nhis-AerAV(GG). AR1-3 correspond to the core peptide sequences from the rhp cluster; AP to the core from the poy cluster; AV to the vep cluster. The yield observed using the GluC generated aeronamide A was more than a 100 fold, when expressed under the new P_{BAD} promoter.

Transformed colonies were picked and grown at induction conditions as described in example 2 followed by protein purification using affinity chromatography (example 3). The purified protein was treated with endoproteinase GluC (example 5) and analyzed by liquid chromatography-mass spectrometry/mass spectrometry (LC-MS/MS) to characterize the sites of modifications (FIGS. 4 and 5). In all cases apart from AerAV, multiple sites carrying C- and N-methylations (catalyzed by aerC and aerE respectively) were localized. Thr1 dehydration (catalyzed by aerF) was observed for AerA, AerA(GG), AerAR1 and AerAR2. To localize epimerizations, ODIS (Orthogonal D₂O-based induction system, example 4) was performed. The precursor peptide sequences were expressed in E. coli followed by expression of the epimerase AerD in a deuterated background. Using this, an alternating pattern of epimerization was observed for all peptide sequences with AerA (21 epimerizations, FIG. 2) and AerAR2 (23 epimerizations) undergoing full epimerization. For AerAR1 and AerAP 16 and 6 epimerizations were localized respectively.

To generate aeronamide A, Nhis-AerA purified from 5.5 L of M. aerodenitrificans was cleaved with Nhis-AerH purified from E. coli (example 5). The reaction mixture was purified using a C-18 solid phase exchange (SPE) column followed by high-pressure liquid chromatography (HPLC) to yield 600 μg of pure aeronamide A (FIG. 6a, example 12).

Aeronamide A showed potent cytotoxic activity against HeLa cells with an IC₅₀ value of 1.48 nM (polytheonamide B: 0.58 nM), but not towards the bacteria and fungi (example 10). To test whether the cytotoxicity is based on a similar pore-forming mechanism as for polytheonamides, an H⁺/Na⁺ ion exchange activity assay was performed on artificial liposomes (examples 7 and 8). Satisfyingly, a similar capability exhibited by polytheonamides for transporting H₊ and Na⁺ ions was induced by aeronamide A (FIG. 6b). The almost picomolar range of activity was unexpected given that aeronamide A lacks the tert-butyl moiety on Thr1 (FIG. 6c), implicated as being a driving factor in polytheonamide cytotoxicity.

1.-19. (canceled)

20. A nucleic acid, comprising a nucleic acid sequence selected from the group consisting of:
(i) a nucleic acid of any one of SEQ ID NOs: 1 (aerC), 3 (aerD), 5 (aerF), or 7 (aerE);
(ii) a nucleic acid sequence of at least 80 or 90% sequence identity with a nucleic acid sequence of (i);
(iii) a nucleic acid sequence that hybridizes to a nucleic acid sequence of (i) or (ii) under stringent conditions;
(iv) a fragment of any of the nucleic acid sequences of (i) to (iii), that hybridizes to a nucleic acid sequence of (i) or (ii) under stringent conditions;
(v) a nucleic acid sequence degenerated with respect to a nucleic acid sequence of any of (i) to (iv);
(vi) a nucleic acid sequence, wherein said nucleic acid sequence is derivable by substitution, addition and/or deletion of at least one nucleic acid of the nucleic acid sequences of (i) to (v) that hybridizes to a nucleic acid sequence of (i) or (ii) under stringent conditions;
(vii) a nucleic acid sequence complementary to the nucleic acid sequence of any of (i) to (vi);
wherein the nucleic acid sequence of any of (i) to (vii),
(a) when based on SEQ ID NO: 1 (aerC) encodes a polypeptide that has cobalamin-dependent rSAM methyltransferase activity;
(b) when based on SEQ ID NO: 3 (aerD) encodes a polypeptide that has rSAM epimerase activity to convert one or more L-amino acid(s) into D-amino acid(s);
(c) when based on SEQ ID NO: 5 (aerF) encodes a polypeptide that has dehydratase activity to dehydrate an N-terminal threonine or serine to an alpha-keto functional group; or
(d) when based on SEQ ID NO: 7 (aerE) and encodes a polypeptide that has asparagine (ASN)N-methyltransferase activity for methylating one or more side chain amines of one or more asparagine(s).

21. The nucleic acid according to claim 20, wherein the nucleic acid comprises a nucleic acid sequence of at least 95% sequence identity with a nucleic acid sequence of (i).

22. The nucleic acid according to claim 20, wherein the nucleic acid comprises a nucleic acid sequence of at least 98% sequence identity with a nucleic acid sequence of (i).

23. The nucleic acid according to claim 20, wherein the nucleic acid sequence of any of (i) to (vii), when based on SEQ ID NO: 1 (aerC), encodes a polypeptide that methylates one or more valine(s) to tert-leucine(s), methylates one or more isoleucine(s), methylates one or more leucine(s), methylates one or more threonine(s), or a combination thereof.

24. A polypeptide selected from the group consisting of:
(i) a polypeptide comprising an amino acid sequence selected from the group consisting of SEQ ID NOs: 2, 4, 6 and 8,
(ii) a polypeptide encoded by a nucleic acid of claim 20;
(iii) a polypeptide having an amino acid sequence identity of at least 70% with the polypeptides of (i) and/or (ii); and
(iv) a functional fragment and/or functional derivative of (i), (ii) or (iii);
wherein the polypeptide of any of (i) to (iv),
(a) when based on an amino acid sequence of SEQ ID NO: 2 (AerC) has cobalamin-dependent rSAM methyltransferase activity;
(b) when based on an amino acid sequence of SEQ ID NO: 4 (AerD) has rSAM epimerase activity to convert one or more L-amino acid(s) into D-amino acid(s);
(c) when based on an amino acid sequence of SEQ ID NO: 6 (AerF) has dehydratase activity to dehydrate an N-terminal threonine or serine to an alpha-keto functional group; or
(d) when based on an amino acid sequence of SEQ ID NO: 8 (AerE) has asparagine (ASN) N-methyltransferase activity for methylating one or more side chain amine(s) of asparagine(s).

25. The polypeptide according to claim 24, wherein polypeptide is a selected from a polypeptide having an amino acid sequence identity of at least 90% with the polypeptide of (i) and/or (ii).

26. The polypeptide according to claim 24, wherein the polypeptide of any of (i) to (iv), when based on an amino acid sequence of SEQ ID NO: 2 (AerC), methylates one or more valine(s) to tert-leucine(s), methylates one or more isoleucine(s), methylates one or more leucine(s), methylates one or more threonine(s), or a combination thereof.

27. An antibody, a functional fragment or functional derivative thereof, or antibody-like binding protein that specifically binds a polypeptide of claim 24.

28. A vector or a plasmid, comprising a nucleic acid according to claim 20.

29. A bacterial host cell comprising a nucleic acid according to claim 20, wherein the host cell expresses one or more polypeptides selected from:
(v) a polypeptide comprising an amino acid sequence selected from the group consisting of SEQ ID NOs: 2, 4, 6 and 8,
(vi) a polypeptide encoded by the nucleic acid of claim 20;
(vii) a polypeptide having an amino acid sequence identity of at least 70% with the polypeptides of (i) and/or (ii); and
(viii) a functional fragment and/or functional derivative of (i), (ii) or (iii);
wherein the polypeptide of any of (i) to (iv),
(e) when based on an amino acid sequence of SEQ ID NO: 2 (AerC) has cobalamin-dependent rSAM methyltransferase activity;
(f) when based on an amino acid sequence of SEQ ID NO: 4 (AerD) has rSAM epimerase activity to convert one or more L-amino acid(s) into D-amino acid(s);
(g) when based on an amino acid sequence of SEQ ID NO: 6 (AerF) has dehydratase activity to dehydrate an N-terminal threonine or serine to an alpha-keto functional group; or
(h) when based on an amino acid sequence of SEQ ID NO: 8 (AerE) has asparagine (ASN) N-methyltransferase activity for methylating one or more side chain amine(s) of asparagine(s).

30. The bacterial host cell according to claim 29, wherein the bacterial host cell produces cobolamin, is an E. coli host cell, or a combination thereof.

31. The bacterial host cell according to claim 29, wherein the bacterial host cell is a Microvirgula aerodenitrificans host cell, wherein the host cell expresses at least one heterologous polypeptide for enzymatic modification and modifies the at least one heterologous polypeptide.

32. The bacterial host cell according to claim 31, wherein the host cell expresses at least one of:
(i) at least one polypeptide based on amino acid sequence SEQ ID NO: 2 (AerC);
(ii) at least one polypeptide based on amino acid sequence SEQ ID NO: 4 (AerD),
(iii) at least one polypeptide based on amino acid sequence SEQ ID NO: 6 (AerF);
(vi) at least one polypeptide based on amino acid sequence SEQ ID NO: 8 (AerE); or
(vii) a combination thereof,
with the proviso that expression of polypeptide (v) requires the expression of polypeptide (ii).

33. A bacterial host cell of claim 30, wherein the host cell is an Escherichia coli host cell and wherein the host cell expresses at least one of:
(i) at least one polypeptide based on amino acid sequence SEQ ID NO: 2 (AerC);
(ii) at least one polypeptide based on amino acid sequence SEQ ID NO: 4 (AerD)
(iii) at least one polypeptide based on amino acid sequence SEQ ID NO: 6 (AerF);
(vi) at least one polypeptide based on amino acid sequence SEQ ID NO: 8 (AerE); or
(vii) a combination thereof,
with the proviso that (a) expression of polypeptide (iv) requires the expression of polypeptide (ii) and (b) expression of (i) requires bacterial production or supplement of cobalamin.

34. The host cell according to claim 31, wherein the Microvirgula aerodenitrificans host cell expresses a heterologous polypeptide for enzymatic modification selected from the group of polypeptide precursors of boceprevir, telapevir, glecaprevir, atazanavir, vancomycin, colistin, teixobactin, bacitracin, gramicidin A-D, goserelin, leuprolide, nateglidine, octreotide, thiostreptons, bottromycins polymyxin, actinomycin, nisin, protegrin, dalbavancin, daptomycin, enfurvirtide, oritavancin, teicoplanin and guavanin 2.

35. The host cell according to claim 31, wherein the Microvirgula aerodenitrificans host cell expresses a heterologous polypeptide for enzymatic modification encoded by a nucleic acid sequence comprised in the aerA cluster of Microvirgula aerodenitrificans and encompassing the nucleic acid sequence of Seq. ID. NO.: 9 or a nucleic acid sequence hybridizing thereto under stringent conditions.

36. A composition comprising at least one nucleic acid according to claim 20.

37. A method for producing and modifying a heterologous (poly)peptide in a Microvirgula aerodenitrificans cell or an E. coli cell, comprising the steps of
(i) providing a Microvirgula aerodenitrificans host cell or an E. coli host cell functionally expressing
a. at least one polypeptide enzyme according to claim 29; and
b. at least one heterologous (poly)peptide of interest; and
(ii) co-expressing the at least one polypeptide enzyme according to claim 29 and the at least one heterologous (poly)peptide of interest;
wherein the at least one polypeptide enzyme according to claim 29 is capable of catalyzing at least one modification in the heterologous (poly)peptide of interest.

38. The method of claim 37, comprising the steps of
(i) providing a Microvirgula aerodenitrificans or a cobalamin-producing E. coli host cell, functionally expressing
a. at least one Cbl-dependent rSAM polypeptide enzyme; and
b. at least one heterologous (poly)peptide of interest; and
(ii) co-expressing the at least one Cbl-dependent rSAM enzyme and the at least one heterologous (poly)peptide;
wherein the at least one Cbl-dependent rSAM enzyme methylates one or more valine(s) to tert-leucine(s), methylates one or more isoleucine(s), methylates one or more leucine(s), methylates one or more threonine(s), or a combination thereof, in the at least one heterologous (poly)peptide of interest.

39. The method according to claim 37, wherein the method further comprises at least one of:
(iii) co-expressing one or more further enzymes for modifying the at least one heterologous (poly)peptide of interest; or
(iv) at least partially purifying the so-modified heterologous (poly)peptide.

40. The method according to claim 37, wherein the one or more further enzymes for modifying the heterologous (poly)peptide(s) in step (iii) are selected from the polypeptides according to claim 5.

41. The method according to claim 38, wherein the one or more further enzymes for modifying the heterologous (poly)peptide(s) in step (iii) are selected from the group consisting of PoyB, PoyC (rSAM C-methyltransferases), OspD, AvpD, PlpD, PoyD (epimerases), PlpXY (n-amino acid incorporation), and PtsY (S-methyltransferase).

42. The method according to claim 37, wherein the at least one heterologous (poly)peptide is selected from the group consisting of polypeptide precursors of boceprevir, telapevir, glecaprevir, atazanavir, vancomycin, colistin, teixobactin, bacitracin, gramicidin A-D, goserelin, leuprolide, nateglidine, octreotide, thiostreptons, bottromycins polymyxin, actinomycin, nisin, protegrin, dalbavancin, daptomycin, enfurvirtide, oritavancin, teicoplanin, and guavanin 2.

43. The method according to claim 37, wherein at least one of (i) the heterologous (poly)peptide of interest, the polypeptide enzyme(s) according to claim 5, the one or more further enzymes for modifying the heterologous (poly)peptide(s), or a combination thereof, are present in the form of host-integrated DNA and/or in the form of a plasmid.

44. A polypeptide comprising a posttranslational modification selected from the group consisting of
(i) a methylation of one or more valine(s) to tert-leucine(s), a methylation of one or more isoleucine(s), a methylation of one or more leucine(s), a methylation of one or more threonine(s);
(ii) a conversion of one or more L-amino acid(s) into D-amino acid(s);
(iii) a hydrolyzation of an N-terminal dehydro-threonine or -serine to an alpha-keto functional group; and
(iv) a methylation of one or more side chain amine(s) of asparagine(s),
wherein the polypeptide is obtained by a method according to claim 37.


SEQUENCE LISTING (25 sequences)

SEQ ID NO: 1 - DNA, 2085 bp, Microvirgula aerodenitrificans
atgtgggaaagcaacggattgccggaagcgcctgatgactccccggccctgcacgccggcgagtcctgttccggcggcatcgacatggtgttcgtgtgcatgccgtacgccgccgtggaacgtccgtcgctggccctcggcacgctgactgcggtgcttgagcgcgaaggtctgtccagccgggcgatctacgccaatctcgaatttgccgcccgcgtcggccggcaggcctatgaagtcgtcaacaactccgaaatcacgctccagctcggcgaatggacgttctccgaagcggtattcggacagcagggcgacatcgacgcctttatcaagggcctggtcagctgtggctacaccgagaccggcctgcgggagctgctgcagggcctgcgcctcgaggccgcccgctatctcgacgagctggcgcagcgtgtcctggccctgcagccgcgcattgtcggctgcacgtccatgttccagcagcactgcgcgtccctggggctgcttcagcgcatccgggcgcagtccccgggtacggtgaccatgctgggcggcgcaaactgcgaaggggaaatgggcgcggccacccacaggcaatatccatgggtcgactttgtcgtgtccggagaagccgacaaattgctgcccgaactctgcagacgcattctggcccggggcgcgggcattccggtccatcacctgcccgagggcgtgctcggcccggcctcgcgccgcgtccttgtcgttgcgggcgccgcggcagcaccggcggtcggtcgggcttcgattaccgatctcgacgaattgccgattccgaacttcgacgactatttcgagcagctgcaagcctcgccgctgcacggctacgtgattcccggcctgctgatcgaaacctcccgcggttgctggtggggcgccaagcaccactgcacgttctgcggcctgaacggttcgggcatggcgtttcgcgccaagtcgcaagcgcgagtccagcaagaagtcagccagctggcggcgcgctaccggctcaagcggttcatggcggtcgacaatattctcgacaacaaatacttctcccaggttctgccctttctggccgaagccggcgacatgctgtggttctacgaaaccaaggccaacctcacccgcacccaggtcagcttgctgtcgcaggccggggtgcgctggattcagccgggtatcgaggcgatggacgacggcctgctgaagctgctgcgcaagggctgctcgaccgttatcaatgtgcagctgctgaagtgggcctacgactacggcgtgtgggtgatgtggaaccacctgcacggcgcgccgggcgaagatcccgagtggtatgagcacattgccgactggctgccgctgattgcgcacctgcagccgccatcgggcggctccatgacccgcatccgcttcgaccgtttcagcccctacttcaatgaacaggccgacttcgccctggatctcaagccctgctggggctatggccaggtctatcccgtgccggaaaagcagctcgaacagcaggcctacttcttccgcaatgacggccactccgccccgacgccgacccggctggcggcaatgctgagcgagtgggccacccgcttctacgcgccgactaccagggcaaccacgctgccccggcggagtgatgacgccccggtgctggcctgggtcgccagcggctaccggcagacggtccgcgatacccggccctgtgccgtcgggtcgctgcacgaactcagcgagctggaaggacaggtctgtcatgccctcgacagtgcgcagcatctgcagggtctggtgcaggccctgcgccatgccggcagcacggtcccggaaagggaaatcggctccgcgctgcaacggctggtcgatctgaaaatcattgccgaattcaacggcaagttcctttgcctggtcaccagcgagaacccggtgccctacaagtcgttctccgagtttgccggcggcatgttcagccttacccccacacaccggacaccgccgaagcccgaaaccccatgggatgtttccttgagggagttgtttgtctcttccacgtaa

SEQ ID NO: 2 - protein, 694 aa, Microvirgula aerodenitrificans
MWESNGLPEAPDDSPALHAGESCSGGIDMVFVCMPYAAVERPSLALGTLTAVLEREGLSSRAIYANLEFAARVGRQAYEVVNNSEITLQLGEWTFSEAVFGQQGDIDAFIKGLVSCGYTETGLRELLQGLRLEAARYLDELAQRVLALQPRIVGCTSMFQQHCASLGLLQRIRAQSPGTVTMLGGANCEGEMGAATHRQYPWVDFVVSGEADKLLPELCRRILARGAGIPVHHLPEGVLGPASRRVLVVAGAAAAPAVGRASITDLDELPIPNFDDYFEQLQASPLHGYVIPGLLIETSRGCWWGAKHHCTFCGLNGSGMAFRAKSQARVQQEVSQLAARYRLKRFMAVDNILDNKYFSQVLPFLAEAGDMLWFYETKANLTRTQVSLLSQAGVRWIQPGIEAMDDGLLKLLRKGCSTVINVQLLKWAYDYGVWVMWNHLHGAPGEDPEWYEHIADWLPLIAHLQPPSGGSMTRIRFDRFSPYFNEQADFALDLKPCWGYGQVYPVPEKQLEQQAYFFRNDGHSAPTPTRLAAMLSEWATRFYAPTTRATTLPRRSDDAPVLAWVASGYRQTVRDTRPCAVGSLHELSELEGQVCHALDSAQHLQGLVQALRHAGSTVPEREIGSALQRLVDLKIIAEFNGKFLCLVTSENPVPYKSFSEFAGGMFSLTPTHRTPPKPETPWDVSLRELFVSST

SEQ ID NO: 3 - DNA, 1536 bp, Microvirgula aerodenitrificans
atgcccacgccacaagggacaagctacgccgaaccgcaacaccctgccgccgacgccacgccggactggccgcccatcacgatcgggcaggccaagcgcgttctggaatggtggtcttcgtctgcggtgttccgtgaactggtggcgaccgatccggaacgcgccggccgcgactacaaactgggcttcagtcccgaactgatccgtccgctgtgggacgaccgctatcacctcgacgccgccaacaaggatcgcccgcaacacccgatcgttgccgagtaccgggcttattaccacaccaagacgcagtggcgcgacgaggtcaaaagggagtgcgccccggacgagccccgcctgaaaacctggcgtacccgccagatcgcccgcaatgcgatggaaaacggtctttacgacaacagcatcattcactcgccgctggccatcgagctcagcgatggctgttcggtcggttgctggttctgcggcgtcggcgcgaccaggtttgttgagacctgggactacaccgaggaaaacgccacgctgtggcgcggcgtgctgagcgtattgcacgacaagatcggcgatgccagcaaatgggggttctgctactgggccaccgacccgctggacaatccggactacgagcacttcgccagcgattttgccgatatcaccggcatgttcccgcagaccaccaccgcccagggccacaaggacccggaacgggtgctcaagctgctcaggctgtccgaatcgcgcggctgcaaggtcaaccgcttctcggtcctgaccgaatcgctgctgcgccggattcacgatgcatacacggcagatgagctgacccaggtcgagattgtcgcccagatgcgtgacgctaccgtgccgaaggccgacgccggctcattccgggtaaaggccaggaagactgccaatgtcgtggagcgggaaaagaaaaaactgattccgatcgccgtgtccgagaacgggacggaagacagcgacaagcccgcgctgaccatgcagcagccgggcaccatcgcctgcgttaccggcttcctgctcaatatggtccggcgctcggtcaagctgatcagtccctgccgtgcttccgagcaatggccgctcggttacatcgtgttcgaggaatgcacgttcaccgacgccgccgacctggaacgcaagatcgaagccatgatcgagacccacatgccgcaggagctcacgccggacgacccgatccagctcaatccgagcttccgcctggagccggtagccaacggtttccgcgtgctttccgacctgagctgcatcgagttctcccgccccacccctgacctggtcgagtacctcggcgcgctgggcgagcacgtgacatcaggcaagcgtacggccggtgaaatcgccgtgtcctcgctgttctgctatggcgtgccggagacaaacaccctgagcaccctcggcgcgatgctgaacctcgggatcctggtcgatgccaggggccgcattaccggccaatcagcagtggaagcccaatga

SEQ ID NO: 4 - protein, 511 aa, Microvirgula aerodenitrificans
MPTPQGTSYAEPQHPAADATPDWPPITIGQAKRVLEWWSSSAVFRELVATDPERAGRDYKLGFSPELIRPLWDDRYHLDAANKDRPQHPIVAEYRAYYHTKTQWRDEVKRECAPDEPRLKTWRTRQIARNAMENGLYDNSIIHSPLAIELSDGCSVGCWFCGVGATRFVETWDYTEENATLWRGVLSVLHDKIGDASKWGFCYWATDPLDNPDYEHFASDFADITGMFPQTTTAQGHKDPERVLKLLRLSESRGCKVNRFSVLTESLLRRIHDAYTADELTQVEIVAQMRDATVPKADAGSFRVKARKTANVVEREKKKLIPIAVSENGTEDSDKPALTMQQPGTIACVTGFLLNMVRRSVKLISPCRASEQWPLGYIVFEECTFTDAADLERKIEAMIETHMPQELTPDDPIQLNPSFRLEPVANGFRVLSDLSCIEFSRPTPDLVEYLGALGEHVTSGKRTAGEIAVSSLFCYGVPETNTLSTLGAMLNLGILVDARGRITGQSAVEAQ

SEQ ID NO: 5 - DNA, 1830 bp, Microvirgula aerodenitrificans
atggccggtgcgcgggcgcccttgctgcacgaactgttgcacgccgcaccggcggaaaccggcgcgcggatgagcgatacgctgtatcgccgctggccggcgctgctgggcaccgacacgctgtccaggcggctggactggggctatgaaggccccgatccagccgatgccccaccgtgggaagacatgctggcggcggcgctgtccgcaccggacgacgccgccctgcccggcagtctcgaccccatcgggccgattccgttcgaggaagcgctgctgcccttcgtcgccgccgcacgcaccgggctggagcgcgaagcgggtacggcactcggcatctgctccccggcggcgcgagcggcgctggaacgccatctgttcgcgctgctctccgtcgttgccactccggtgctcggttccgcgttttccttgcagcgcgcgctggccggctcattgccctggctgcccccccggggtcgccagaactacgaagcctttgtcgcttcgctgcaggccggtggtctgcgcctgctgctggcagactatcccgcactggcgcgcctgctggcgactgtggcccggggatgggtgctggcccacggccggcttctgcagcggctggaaagggactggcaccggatcgtcgccacgttcggcttccccgccgccgactgtcagctggacgggatcacggccggctgctccgacccccatgacggggggcagagcgtaaccgttctgcacctgtcgaatggccggcgcctggtctacaagcccaggtcgctggagatggagaacgggctggcgcaactgctcgactgggccggtcgcaccggctttccgtggccatttcgcaccgtggcgttgctgcccggcgagggctatggctggatggagttcgtcgaggcggcgccctgcgacgacgaggcggccgtcgggcgtttccatgcccgctcaggcggactgttctgcctgtggtggctgctgcagggaaccgacgtccaccacgaaaacctgatcgccagcggggagtatcccgtgatcgtcgatgccgagaccctgctgcatccccgcccctttcccggcgtgattcaccagttgggcccgggcgccggttacggtgcaagcccggaggatgactttgcccgggcactgctggagagcggtttcctggctaccggcaaggcgctcgatctgtcggcctggggacaggccggggacggggccacgccgtttcaggtcgccagctgccaggccatcaattccgatgccatgaccatggcgcacgagacctttcatgtcgcaccgcgtcccaacctgccggtgctgcacggccagccccggcaggcggcggcgcatgccggggcgatcgtcgacgggttcacggcgatgtaccggctggtcgtgcagcaccgccatgactttctgaccctgcttgatggcttcgccggttgcagcgggcgctttgtcgcgcgcgcgaccaatacctacgggctgctgctgcatgccggcctgcaaccggaatggctgcgcgaaggccctgcccgcggcgtgcttttcgagcgcctgcgccaggcggcgctggcggccccggcacgccctgcgtgctggcccctgctcgacgccgaactggcggcgctggaacggctcgacatcccccgcctgtcctgccgctgcgacctgcccgcgccctgctggccggcgccactggatcaggcccgtgccagagttgcgagtgcctcgctgcctgatctcgaccggcatgcggcagcactccggcaagcactgacaccccagaccgcctctccccgccaccctcacccagcctga

SEQ ID NO: 6 - protein, 609 aa, Microvirgula aerodenitrificans
MAGARAPLLHELLHAAPAETGARMSDTLYRRWPALLGTDTLSRRLDWGYEGPDPADAPPWEDMLAAALSAPDDAALPGSLDPIGPIPFEEALLPFVAAARTGLEREAGTALGICSPAARAALERHLFALLSVVATPVLGSAFSLQRALAGSLPWLPPRGRQNYEAFVASLQAGGLRLLLADYPALARLLATVARGWVLAHGRLLQRLERDWHRIVATFGFPAADCQLDGITAGCSDPHDGGQSVTVLHLSNGRRLVYKPRSLEMENGLAQLLDWAGRTGFPWPFRTVALLPGEGYGWMEFVEAAPCDDEAAVGRFHARSGGLFCLWWLLQGTDVHHENLIASGEYPVIVDAETLLHPRPFPGVIHQLGPGAGYGASPEDDFARALLESGFLATGKALDLSAWGQAGDGATPFQVASCQAINSDAMTMAHETFHVAPRPNLPVLHGQPRQAAAHAGAIVDGFTAMYRLVVQHRHDFLTLLDGFAGCSGRFVARATNTYGLLLHAGLQPEWLREGPARGVLFERLRQAALAAPARPACWPLLDAELAALERLDIPRLSCRCDLPAPCWPAPLDQARARVASASLPDLDRHAAALRQALTPQTASPRHPHPA

SEQ ID NO: 7 - DNA, 1155 bp, Microvirgula aerodenitrificans
atgaccccgccactcgccaccaccattgaccggctccgcgactaccttgaccgggtcggcttccagcagatctacaaatacattgtcgcggtcaaccattacgccgtgacgccggcgctgatcacgcgcaacactgccgccagtgtccaccacttcttcgatagccggctgggcggcagggcggaattcgccctgttgcagtgcctgatgaccgggcgtccggccgagcatgcagcgctgccggacaaggaccgggcgctggccgatgcgctggtgacggccggcctgctgcgggccagtccggatgggcgggaagtgtccggcgcggaccggcagctgatttcggccttcggcgtggatctgctgatcgaccgccgcattcatttcggcggcgaagtccacgaggtgtatatcggtcccgacagctactggatgctgtattacatcaatgcttccggtattgcccgcacgcatcgcgccgtcgacctgtgcaccggcagcggcattgccgcgctgtatctgtcgctgtttaccgatcatgttctggcgaccgatatcggcgatgtgccgctggcgctggtcgagataaaccgccgcctgaaccggcgcgacgccggcacgatggagatccggcgcgagaacctgaacgacacgctggatggccgtgaacgcttcgacctgctcacctgcaacccgcctttcgtggccttccctcccggctacagcggcacgctctattcgcagggcaccggcgtcgacggactcggctacatgcgcgacatcgtcggccgcctgccggaagtgctcaatcccggcggttctgcctacctcgtggcggatctctgcggcgatgcgcacggcccgcacttcctgggtgagctggagagcatggtcaccgggcacggcatgcgcatcgaggcgttcatcgaccatgtcctgccggcctcggcccaggtcggcccgatctcggacttcctgagacacgcagccgggctgcctgcggacaccgacatcgcggcagacgtgcaggcgttccagcgcgagacgctgcgcgccgactactactacctgacgacgatccgcctgcaaacggccgcgcagaaccccggactgcgcatgctgcgacgcgacccgctccccggggccgggacgtga

SEQ ID NO: 8 - protein, 384 aa, Microvirgula aerodenitrificans
MTPPLATTIDRLRDYLDRVGFQQIYKYIVAVNHYAVTPALITRNTAASVHHFFDSRLGGRAEFALLQCLMTGRPAEHAALPDKDRALADALVTAGLLRASPDGREVSGADRQLISAFGVDLLIDRRIHFGGEVHEVYIGPDSYWMLYYINASGIARTHRAVDLCTGSGIAALYLSLFTDHVLATDIGDVPLALVEINRRLNRRDAGTMEIRRENLNDTLDGRERFDLLTCNPPFVAFPPGYSGTLYSQGTGVDGLGYMRDIVGRLPEVLNPGGSAYLVADLCGDAHGPHFLGELESMVTGHGMRIEAFIDHVLPASAQVGPISDFLRHAAGLPADTDIAADVQAFQRETLRADYYYLTTIRLQTAAQNPGLRMLRRDPLPGAGT

SEQ ID NO: 9 - DNA, 300 bp, Microvirgula aerodenitrificans
atgactacgactacaccggccagcacccaggttccgcaaacccggcgcgatctggaaacccacatcatcaccaaggcctggaaggatcccgagtacaaggcccagctgctcaaggacccgaaggcggcgctgcaggatgcgctcaagagcattgacccgtccctctccctgcccgactcgctgcaggtccaggtgcacgaggagaacgccaacctgttccaccttgtgctgccgcgcaatccgagcgagatctcgctcgccgaggtggtaggcgacaaccttgaagccgtggcaccgcaa

SEQ ID NO: 10 - protein, 100 aa, Microvirgula aerodenitrificans
MTTTTPASTQVPQTRRDLETHIITKAWKDPEYKAQLLKDPKAALQDALKSIDPSLSLPDSLQVQVHEENANLFHLVLPRNPSEISLAEVVGDNLEAVAPQ

SEQ ID NO: 11 - protein, 54 aa, Candidatus Entotheonella factor
QAAGGTGIGVVVAVVAGAVANTGAGVNQVAGGNINVVGNINVNANVSVNMNQTT

SEQ ID NO: 12 - protein, 51 aa, Microvirgula aerodenitrificans
AVAPQTIAVVLVAVVGAAAAAVVTYLGAANVVGAANGTVTANAVANTNAVA

SEQ ID NO: 13 - protein, 55 aa, Rhodospirillaceae bacterium BRH_c57
AVAPQTIAVVVAVVGIGVVAGNTLGVVNNVGAGNAVAAGNVATTGNAVANTNVIA

SEQ ID NO: 14 - protein, 53 aa, Rhodospirillaceae bacterium BRH_c57
AVAPQTIAVVVAALGVVVANTLGAVNNVGAGNAVTVGNVATTGNAVANSTSVS

SEQ ID NO: 15 - protein, 38 aa, Rhodospirillaceae bacterium BRH_c57
AVAPQTIAVVTNGVGVCAVVTGPVTIAYPTNVVTCVVA

SEQ ID NO: 16 - protein, 52 aa, Verrucomicrobia bacterium SCGC AAA164-I21
AVAGGVAAIAVFVVGVVAVAVGGTVTVAVNINAAVNVHTVVNAVKGANESPW

SEQ ID NO: 17 - protein, 46 aa, Microvirgula aerodenitrificans
TIAVVLVAVVGAAAAAVVTYLGAANVVGAANGTVTANAVANTNAVA

SEQ ID NO: 18 - protein, 51 aa, Microvirgula aerodenitrificans
AVAGGTIAVVLVAVVGAAAAAVVTYLGAANVVGAANGTVTANAVANTNAVA

SEQ ID NO: 19 - protein, 55 aa, Microvirgula aerodenitrificans; identical to SEQ ID NO: 13

SEQ ID NO: 20 - protein, 53 aa, Microvirgula aerodenitrificans; identical to SEQ ID NO: 14

SEQ ID NO: 21 - protein, 54 aa, Microvirgula aerodenitrificans
AVAPQTGIGVVVAVVAGAVANTGAGVNQVAGGNINVVGNINVNANVSVNMNQTT

SEQ ID NO: 22 - protein, 33 aa, Microvirgula aerodenitrificans
TIAVVTNGVGVCAVVTGPVTIAYPTNVVTCVVA

SEQ ID NO: 23 - DNA, 102 bp, Microvirgula aerodenitrificans
accatcgccgtcgtcaccaacggcgtcggcgtgtgcgcagtcgtgaccggcccggtgaccatcgcctatcccacgaacgtggtgacttgcgtcgtcgcctga

SEQ ID NO: 24 - protein, 38 aa, Microvirgula aerodenitrificans; identical to SEQ ID NO: 15

SEQ ID NO: 25 - DNA, 1215 bp, Artificial Sequence
  Arabinose regulator sequence, arabinose promoter sequence and aer promoter RBS
ttatgacaacttgacggctacatcattcactttttcttcacaaccggcacgaaactcgctcgggctggccccggtgcattttttaaatactcgcgagaaatagagttgatcgtcaaaaccaacattgcgaccgacggtggcgataggcatccgggtagtgctcaaaagcagcttcgcctgactaatgcgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaacccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccaccattcagagaagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttaggagagtgcggg